(19) United States
(12) Patent Application Publication
(10) Pub. No.: US 2023/0076689 A1
(43) Pub. Date: Mar. 9, 2023

(54) FLOW CELL IMAGE SENSOR ARRANGEMENT WITH REDUCED CROSSTALK

(71) Applicant: ILLUMINA, INC., San Diego, CA (US)

(72) Inventors: Mohsen Rezaei, Brown Deer, WI (US); Craig Hetherington, Foster City, CA (US); Arvin Emadi, Hayward, CA (US); Stanley Hong, Palo Alto, CA (US)

(21) Appl. No.: 17/896,176

(22) Filed: Aug. 26, 2022

Related U.S. Application Data
(60) Provisional application No. 63/237,640, filed on Aug. 27, 2021.

(51) Int. Cl. G01N 15/14; G02B 5/20; H01L 27/146

(52) U.S. Cl. CPC G01N 15/1436; G02B 5/201; H01L 27/14623; G01N 21/77

(57) ABSTRACT

An apparatus includes a flow cell body with an array of reaction sites positioned along a floor of a channel. An optical filter layer is positioned under the floor of the channel and includes at least a portion spanning uninterruptedly along a length corresponding to the length of the array of reaction sites. Imaging regions are positioned under the optical filter layer. Each imaging region is positioned directly under a corresponding reaction site. The optical filter layer is configured to permit one or more selected wavelengths of light to pass from each reaction site to the imaging region forming a sensing pair with the reaction site. The optical filter layer is configured to reduce transmission of excitation light directed toward the reaction sites; and to reduce transmission of light emitted from each reaction site to imaging regions not forming a sensing pair with the reaction site.

### PRIORITY

This application claims priority to U.S. Provisional Pat. App. No. 63/237,640, entitled “Flow Cell Image Sensor Arrangement with Reduced Crosstalk,” filed Aug. 27, 2021, the disclosure of which is incorporated by reference herein, in its entirety.

### BACKGROUND

Aspects of the present disclosure relate generally to biological or chemical analysis and more particularly to systems and methods using image sensors for biological or chemical analysis.

Various protocols in biological or chemical research involve performing a large number of controlled reactions on local support surfaces or within predefined reaction chambers. The designated reactions may then be observed or detected, and subsequent analysis may help identify or reveal properties of chemicals involved in the reaction. For example, in some multiplex assays, an unknown analyte having an identifiable label (e.g., fluorescent label) may be exposed to thousands of known probes under controlled conditions. Each known probe may be deposited into a corresponding well of a flow cell channel. Observing any chemical reactions that occur between the known probes and the unknown analyte within the wells may help identify or reveal properties of the analyte. Other examples of such protocols include known DNA sequencing processes, such as sequencing-by-synthesis (SBS) or cyclic-array sequencing.

In some conventional fluorescent-detection protocols, an optical system is used to direct an excitation light onto fluorescently-labeled analytes and to also detect the fluorescent signals that may emit from the analytes. Such optical systems may include an arrangement of lenses, filters, and light sources. In other detection systems, the controlled reactions occur immediately over a solid-state imager (e.g., charged-coupled device (CCD) or a complementary metal-oxide-semiconductor (CMOS) detector) that does not require a large optical assembly to detect the fluorescent emissions.

In some devices that provide fluorescent detection from several wells or reaction sites, there may be a risk of crosstalk, where a sensor corresponding to one well or reaction site undesirably receives light from either another well or reaction site or some other source. It may therefore be desirable to include features that eliminate or otherwise reduce the risk of such crosstalk. It may also be desirable to provide such crosstalk reduction features without undesirably increasing the manufacturing cost or complexity of the device.

### SUMMARY

Described herein are devices, systems, and methods for reducing or eliminating crosstalk within a flow cell, which may be encountered in systems that perform optical analysis, such as bioassay systems.

An implementation relates to an apparatus that includes a flow cell body defining a channel to receive fluid. The channel has a floor extending along a length of the flow cell body. The apparatus further includes a plurality of reaction sites positioned along the floor of the channel. The plurality of reaction sites form an array along a length of the floor of the channel. The apparatus further includes an optical filter layer positioned under the floor of the channel. The optical filter includes at least a portion spanning uninterruptedly along a length corresponding to the length of the array of reaction sites. The apparatus further includes a plurality of imaging regions positioned under the optical filter layer. Each imaging region of the plurality of imaging regions is positioned directly under a corresponding reaction site, such that each reaction site and corresponding imaging region cooperate to form a sensing pair. The optical filter layer is configured to permit one or more selected wavelengths of light to pass from each reaction site to the imaging region forming a sensing pair with the reaction site. The optical filter layer is configured to reduce transmission of excitation light directed toward the plurality of reaction sites. The optical filter layer is further configured to reduce transmission of light emitted from each reaction site to imaging regions not forming a sensing pair with the reaction site.

In some implementations of an apparatus, such as that described in the preceding paragraph of this summary, the floor of the channel defines a plurality of wells, the plurality of wells providing the plurality of reaction sites.

In some implementations of an apparatus, such as any of those described in any of the preceding paragraphs of this summary, the plurality of wells include nanowells.

In some implementations of an apparatus, such as any of those described in any of the preceding paragraphs of this summary, the flow cell body defines a plurality of channels, the channels being oriented parallel with each other, each channel of the plurality of channels having a floor with a plurality of reaction sites.

In some implementations of an apparatus, such as any of those described in any of the preceding paragraphs of this summary, the plurality of channels form an array along a width of the flow cell body, the optical layer including at least a portion spanning uninterruptedly along a width corresponding to the width of the array of channels.

In some implementations of an apparatus, such as any of those described in any of the preceding paragraphs of this summary, the apparatus further includes a plurality of imaging sensors, each imaging sensor forming a corresponding imaging region of the plurality of imaging regions.

In some implementations of an apparatus, such as any of those described in any of the preceding paragraphs of this summary, each imaging sensor includes a photodiode.

In some implementations of an apparatus, such as any of those described in any of the preceding paragraphs of this summary, the apparatus further includes an imaging chip, the imaging chip spanning along a length corresponding to the length of the array of reaction sites, the imaging chip defining the plurality of imaging regions.

In some implementations of an apparatus, such as any of those described in any of the preceding paragraphs of this summary, the imaging sensor defines a plurality of photodiodes, each imaging region of the plurality of imaging regions being defined by one or more photodiodes of the plurality of photodiodes.

In some implementations of an apparatus, such as any of those described in any of the preceding paragraphs of this summary, the imaging chip includes a CMOS chip.

In some implementations of an apparatus, such as any of those described in any of the preceding paragraphs of this summary, the apparatus further includes a light source, the light source being configured to emit light at an excitation wavelength, the excitation wavelength being configured to cause one or more fluorophores in the reaction sites to fluoresce at an emission wavelength.

In some implementations of an apparatus, such as any of those described in any of the preceding paragraphs of this summary, the optical filter layer substantially prevents transmission of light at the excitation wavelength to the plurality of imaging regions.

In some implementations of an apparatus, such as any of those described in any of the preceding paragraphs of this summary, the optical filter absorbs light at the excitation wavelength.

In some implementations of an apparatus, such as any of those described in any of the preceding paragraphs of this summary, the optical filter layer absorbs at least some light at the emission wavelength.

In some implementations of an apparatus, such as any of those described in any of the preceding paragraphs of this summary, the optical filter layer reduces transmission of light from each reaction site to imaging regions not forming a sensing pair with the reaction site by inducing loss in light transmitted from the reaction sites.

In some implementations of an apparatus, such as any of those described in any of the preceding paragraphs of this summary, the apparatus further includes a plurality of shields, each shield of the plurality of shields to block optical rays between a corresponding reaction site and an imaging region of the plurality of imaging regions that does not form a sensing pair with the corresponding reaction site.

In some implementations of an apparatus, such as any of those described in any of the preceding paragraphs of this summary, each shield of the plurality of shields being aligned with a corresponding sensing pair.

In some implementations of an apparatus, such as any of those described in any of the preceding paragraphs of this summary, the optical filter layer extends along a first height between the floor of the channel and the plurality of imaging regions, the plurality of shields extending along a second height between the floor of the channel and the plurality of imaging regions, the first height being greater than the second height such that the plurality of shields extend along only a portion of the first height.

In some implementations of an apparatus, such as any of those described in any of the preceding paragraphs of this summary, the plurality of shields extend from an underside of the floor, the plurality of shields having lower ends vertically terminating within the optical filter layer.

In some implementations of an apparatus, such as any of those described in any of the preceding paragraphs of this summary, the plurality of shields extend from an upper side of the plurality of imaging regions, the plurality of shields having upper ends vertically terminating within the optical filter layer.

In some implementations of an apparatus, such as any of those described in any of the preceding paragraphs of this summary, the optical filter layer permits transmission of light at wavelengths greater than approximately 600 nm.

In some implementations of an apparatus, such as any of those described in any of the preceding paragraphs of this summary, the optical filter layer prevents transmission of light at wavelengths less than approximately 500 nm.

In some implementations of an apparatus, such as any of those described in any of the preceding paragraphs of this summary, the optical filter layer permits transmission of light at wavelengths greater than approximately 600 nm and prevents transmission of light at wavelengths less than approximately 500 nm.

In some implementations of an apparatus, such as any of those described in any of the preceding paragraphs of this summary, the optical filter layer absorbs some light at wavelengths between approximately 500 nm and approximately 600 nm while permitting transmission of some light at wavelengths between approximately 500 nm and approximately 600 nm.

In some implementations of an apparatus, such as any of those described in any of the preceding paragraphs of this summary, the optical filter layer includes a combination of an orange dye and a black dye.

In some implementations of an apparatus, such as any of those described in any of the preceding paragraphs of this summary, the flow cell body includes a cover positioned over the channel.

In some implementations of an apparatus, such as any of those described in any of the preceding paragraphs of this summary, the cover includes glass.

In some implementations of an apparatus, such as any of those described in any of the preceding paragraphs of this summary, the floor includes glass.

In some implementations of an apparatus, such as any of those described in any of the preceding paragraphs of this summary, the imaging regions are integral with the flow cell body.

In some implementations of an apparatus, such as any of those described in any of the preceding paragraphs of this summary, the optical filter layer has a transmittance coefficient ranging from approximately 0.01 to approximately 0.5.

In some implementations of an apparatus, such as any of those described in any of the preceding paragraphs of this summary, the optical filter layer has a transmittance coefficient ranging from approximately 0.2 to approximately 0.4.

In some implementations of an apparatus, such as any of those described in any of the preceding paragraphs of this summary, the optical filter layer and floor cooperate to define a height dimension, the height dimension corresponding to a distance between a top of the floor and a bottom of the optical filter layer. The plurality of reaction sites define a pitch dimension, the pitch dimension corresponding to a distance between a center of one reaction site of the plurality of reaction sites to a center of an adjacent reaction site of the plurality of reaction sites. The height dimension and pitch dimension provide a height-to-pitch ratio ranging from approximately 3 to approximately 5.

In some implementations of an apparatus, such as any of those described in any of the preceding paragraphs of this summary, the height dimension and pitch dimension provide a height-to-pitch ratio of approximately 4.

In some implementations of an apparatus, such as any of those described in any of the preceding paragraphs of this summary, the apparatus lacks any shields between the plurality of reaction sites and the plurality of imaging regions.

In some implementations of an apparatus, such as any of those described in any of the preceding paragraphs of this summary, the optical filter layer has a thickness ranging from approximately 200 nm to approximately 5 μm.

In some implementations of an apparatus, such as any of those described in any of the preceding paragraphs of this summary, the optical filter layer has a thickness of approximately 1 μm.

In some implementations of an apparatus, such as any of those described in any of the preceding paragraphs of this summary, the optical filter layer is separated from each reaction site by a distance ranging from approximately 25 nm to approximately 500 nm.

In some implementations of an apparatus, such as any of those described in any of the preceding paragraphs of this summary, the apparatus further includes a passivation layer interposed between the optical filter layer and the plurality of imaging regions.

In some implementations of an apparatus, such as any of those described in any of the preceding paragraphs of this summary, the passivation layer includes silicon dioxide.

In some implementations of an apparatus, such as any of those described in any of the preceding paragraphs of this summary, the passivation layer having a thickness ranging from approximately 10 nm to approximately 200 nm.

In some implementations of an apparatus, such as any of those described in any of the preceding paragraphs of this summary, the imaging regions are separated from each other by a pitch distance ranging from approximately 0.5 μm to approximately 25 μm.

In some implementations of an apparatus, such as any of those described in any of the preceding paragraphs of this summary, the imaging regions are separated from each other by a pitch distance of approximately 1 μm.

In some implementations of an apparatus, such as any of those described in any of the preceding paragraphs of this summary, the imaging regions are separated from each other by a pitch distance of approximately 2 μm.

In some implementations of an apparatus, such as any of those described in any of the preceding paragraphs of this summary, the optical filter layer includes a first sub-layer of filter material and a second sub-layer of filter material.

In some implementations of an apparatus, such as any of those described in any of the preceding paragraphs of this summary, the first sub-layer of filter material and the second sub-layer of filter material have the same thickness.

In some implementations of an apparatus, such as any of those described in any of the preceding paragraphs of this summary, the apparatus further includes a plurality of rings, the plurality of rings being positioned adjacent to one or both of the first sub-layer of filter material or the second sub-layer of filter material.

In some implementations of an apparatus, such as any of those described in any of the preceding paragraphs of this summary, each ring of the plurality of rings is associated with a corresponding sensing pair of the sensing pairs formed by each reaction site and corresponding imaging region.

In some implementations of an apparatus, such as any of those described in any of the preceding paragraphs of this summary, each ring of the plurality of rings is centered about an axis passing through a center of a reaction site and imaging region of the sensing pair corresponding with the ring.

In some implementations of an apparatus, such as any of those described in any of the preceding paragraphs of this summary, each ring of the plurality of rings includes a metal.

In some implementations of an apparatus, such as any of those described in any of the preceding paragraphs of this summary, the metal includes tungsten or aluminum.

In some implementations of an apparatus, such as any of those described in any of the preceding paragraphs of this summary, each ring of the plurality of rings has a thickness ranging from approximately 25 nm to approximately 100 nm.

In some implementations of an apparatus, such as any of those described in any of the preceding paragraphs of this summary, the plurality of rings includes a first array of rings and a second array of rings. The first array of rings is located at a first vertical position between the reaction sites and the plurality of imaging regions. The second array of rings is located at a second vertical position between the reaction sites and the plurality of imaging regions.

In some implementations of an apparatus, such as any of those described in any of the preceding paragraphs of this summary, the first array of rings is located at an interface between the first sub-layer of filter material and the second sub-layer of filter material.

In some implementations of an apparatus, such as any of those described in any of the preceding paragraphs of this summary, the second array of rings is located between the second sub-layer of filter material and the plurality of imaging regions.

In some implementations of an apparatus, such as any of those described in any of the preceding paragraphs of this summary, the rings of the first array of rings define openings. The openings of the rings of the first array of rings each have a first diameter. The rings of the second array of rings define openings. The openings of the rings of the second array of rings each have a second diameter. The first diameter is different from the second diameter.

In some implementations of an apparatus, such as any of those described in any of the preceding paragraphs of this summary, the first diameter is smaller than the second diameter.

In some implementations of an apparatus, such as any of those described in any of the preceding paragraphs of this summary, the first diameter is approximately 700 nm.

In some implementations of an apparatus, such as any of those described in any of the preceding paragraphs of this summary, the second diameter is approximately 900 nm.

In some implementations of an apparatus, such as any of those described in any of the preceding paragraphs of this summary, the optical filter layer includes ferric oxide.

Another implementation relates to a method of manufacturing a flow cell. The method includes forming an optical filter layer over an imaging layer, the imaging layer extending along a first length, the imaging layer being operable to capture images at the plurality of imaging regions. The optical filter layer extends continuously along the first length. The method further includes positioning a floor over the optical filter layer, the floor extending along the first length of the flow cell, the floor defining a plurality of reaction sites over the optical filter layer, the plurality of reaction sites forming an array along the first length such that the optical filter layer extends continuously along a region under all the reaction sites of the plurality of reaction sites, each reaction site of the plurality of reaction sites being positioned directly over a corresponding imaging region of the plurality of imaging regions such that each reaction site cooperates with a corresponding imaging region to form a sensing pair. The method further includes positioning a cover over the floor, the floor and the cover cooperating to define a fluid channel, the fluid channel extending along the first length. The cover, the floor, the optical filter layer, and the imaging layer cooperate to form at least a portion of a flow cell body. The optical filter layer is configured to permit one or more selected wavelengths of light to pass from each reaction site to the imaging region forming a sensing pair with the reaction site. The optical filter layer is configured to reduce transmission of excitation light directed toward the plurality of reaction sites. The optical filter layer is further configured to reduce transmission of light emitted from each reaction site to imaging regions not forming a sensing pair with the reaction site.

In some implementations of a method, such as that described in the preceding paragraph of this summary, the imaging layer includes a CMOS chip.

In some implementations of a method, such as any of those described in any of the preceding paragraphs of this summary, the imaging regions include CMOS photodiodes of the CMOS chip.

In some implementations of a method, such as any of those described in any of the preceding paragraphs of this summary, the optical filter layer includes a combination of an orange dye and a black dye.

In some implementations of a method, such as any of those described in any of the preceding paragraphs of this summary, the floor includes glass.

In some implementations of a method, such as any of those described in any of the preceding paragraphs of this summary, the floor includes a plurality of nanowells, the plurality of nanowells sites defining the plurality of reaction sites.

In some implementations of a method, such as any of those described in any of the preceding paragraphs of this summary, the cover includes glass.

In some implementations of a method, such as any of those described in any of the preceding paragraphs of this summary, the flow cell body has a second length, the second length being greater than the first length.

In some implementations of a method, such as any of those described in any of the preceding paragraphs of this summary, the fluid channel extends defining a width, the plurality of reaction sites further forming an array across the width of the fluid channel.

In some implementations of a method, such as any of those described in any of the preceding paragraphs of this summary, the optical filter layer extends continuously across the width of the fluid channel.

In some implementations of a method, such as any of those described in any of the preceding paragraphs of this summary, the floor and the cover cooperate to define a plurality of fluid channels, the fluid channels being oriented parallel with each other, the plurality of fluid channels forming an array across a width of the flow cell body.

In some implementations of a method, such as any of those described in any of the preceding paragraphs of this summary, each fluid channel of the plurality of fluid channels contains a corresponding set of reaction sites of the plurality of reaction sites.

In some implementations of a method, such as any of those described in any of the preceding paragraphs of this summary, the optical filter layer extends continuously across the width of the flow cell body.

In some implementations of a method, such as any of those described in any of the preceding paragraphs of this summary, the optical filter layer is configured to reduce transmission of light from each reaction site to imaging regions not forming a sensing pair with the reaction site by inducing loss in light transmitted from the reaction sites.

In some implementations of a method, such as any of those described in any of the preceding paragraphs of this summary, the method further includes forming a plurality of shields within the optical filter layer, each shield of the plurality of shields to block optical rays between a corresponding reaction site and an imaging region of the plurality of imaging regions that does not form a sensing pair with the corresponding reaction site.

In some implementations of a method, such as any of those described in any of the preceding paragraphs of this summary, each shield of the plurality of shields being aligned with a corresponding sensing pair.

In some implementations of a method, such as any of those described in any of the preceding paragraphs of this summary, the optical filter layer extends along a first height between the floor and the imaging layer, the plurality of shields extending along a second height between the floor and the imaging layer, the first height being greater than the second height such that the plurality of shields extend along only a portion of the first height.

In some implementations of a method, such as any of those described in any of the preceding paragraphs of this summary, the plurality of shields extend from an underside of the floor, the plurality of shields having lower ends vertically terminating within the optical filter layer such that a region of the optical layer extends between the lower ends and the imaging layer.

In some implementations of a method, such as any of those described in any of the preceding paragraphs of this summary, the plurality of shields extend from an upper side of the imaging layer, the plurality of shields having upper ends vertically terminating within the optical filter layer such that a region of the optical layer extends between the upper ends and the floor.

In some implementations of a method, such as any of those described in any of the preceding paragraphs of this summary, the optical filter layer is configured to permit transmission of light at wavelengths greater than approximately 600 nm.

In some implementations of a method, such as any of those described in any of the preceding paragraphs of this summary, the optical filter layer is configured to substantially prevent transmission of light at wavelengths less than approximately 500 nm.

In some implementations of a method, such as any of those described in any of the preceding paragraphs of this summary, the optical filter layer is configured to permit transmission of light at wavelengths greater than approximately 600 nm and prevent transmission of light at wavelengths less than approximately 500 nm.

In some implementations of a method, such as any of those described in any of the preceding paragraphs of this summary, the optical filter layer is configured to absorb some light at wavelengths between approximately 500 nm and approximately 600 nm while permitting transmission of some light at wavelengths between approximately 500 nm and approximately 600 nm.

In some implementations of a method, such as any of those described in any of the preceding paragraphs of this summary, the optical filter layer has a transmittance coefficient ranging from approximately 0.01 to approximately 0.5.

In some implementations of a method, such as any of those described in any of the preceding paragraphs of this summary, the optical filter layer has a transmittance coefficient ranging from approximately 0.2 to approximately 0.4.

In some implementations of a method, such as any of those described in any of the preceding paragraphs of this summary, the optical filter layer and floor cooperate to define a height dimension, the height dimension corresponding to a distance between a top of the floor and a bottom of the optical filter layer. The plurality of reaction sites define a pitch dimension, the pitch dimension corresponding to a distance between a center of one reaction site of the plurality of reaction sites to a center of an adjacent reaction site of the plurality of reaction sites. The height dimension and pitch dimension provide a height-to-pitch ratio ranging from approximately 3 to approximately 5.

In some implementations of a method, such as any of those described in any of the preceding paragraphs of this summary, the height dimension and pitch dimension provide a height-to-pitch ratio of approximately 4.

In some implementations of a method, such as any of those described in any of the preceding paragraphs of this summary, the optical filter layer has a thickness ranging from approximately 200 nm to approximately 5 μm.

In some implementations of a method, such as any of those described in any of the preceding paragraphs of this summary, the optical filter layer has a thickness of approximately 1 μm.

In some implementations of a method, such as any of those described in any of the preceding paragraphs of this summary, the optical filter layer is separated from each reaction site by a distance ranging from approximately 25 nm to approximately 500 nm.

In some implementations of a method, such as any of those described in any of the preceding paragraphs of this summary, the method further includes providing a passivation layer interposed between the optical filter layer and the plurality of imaging regions.

In some implementations of a method, such as any of those described in any of the preceding paragraphs of this summary, the passivation layer includes silicon dioxide.

In some implementations of a method, such as any of those described in any of the preceding paragraphs of this summary, the passivation layer having a thickness ranging from approximately 10 nm to approximately 200 nm.

In some implementations of a method, such as any of those described in any of the preceding paragraphs of this summary, the imaging regions are separated from each other by a pitch distance ranging from approximately 0.5 μm to approximately 25 μm.

In some implementations of a method, such as any of those described in any of the preceding paragraphs of this summary, the imaging regions are separated from each other by a pitch distance of approximately 1 μm.

In some implementations of a method, such as any of those described in any of the preceding paragraphs of this summary, the imaging regions are separated from each other by a pitch distance of approximately 2 μm.

In some implementations of a method, such as any of those described in any of the preceding paragraphs of this summary, the optical filter layer includes a first sub-layer of filter material and a second sub-layer of filter material.

In some implementations of a method, such as any of those described in any of the preceding paragraphs of this summary, the first sub-layer of filter material and the second sub-layer of filter material have the same thickness.

In some implementations of a method, such as any of those described in any of the preceding paragraphs of this summary, the method further includes providing a plurality of rings, the plurality of rings being positioned adjacent to one or both of the first sub-layer of filter material or the second sub-layer of filter material.

In some implementations of a method, such as any of those described in any of the preceding paragraphs of this summary, each ring of the plurality of rings is associated with a corresponding sensing pair of the sensing pairs formed by each reaction site and corresponding imaging region.

In some implementations of a method, such as any of those described in any of the preceding paragraphs of this summary, each ring of the plurality of rings is centered about an axis passing through a center of a reaction site and imaging region of the sensing pair corresponding with the ring.

In some implementations of a method, such as any of those described in any of the preceding paragraphs of this summary, each ring of the plurality of rings includes a metal.

In some implementations of a method, such as any of those described in any of the preceding paragraphs of this summary, the metal includes tungsten or aluminum.

In some implementations of a method, such as any of those described in any of the preceding paragraphs of this summary, each ring of the plurality of rings has a thickness ranging from approximately 25 nm to approximately 100 nm.

In some implementations of a method, such as any of those described in any of the preceding paragraphs of this summary, the plurality of rings includes a first array of rings and a second array of rings. The first array of rings is located at a first vertical position between the reaction sites and the plurality of imaging regions. The second array of rings is located at a second vertical position between the reaction sites and the plurality of imaging regions.

In some implementations of a method, such as any of those described in any of the preceding paragraphs of this summary, the first array of rings is located at an interface between the first sub-layer of filter material and the second sub-layer of filter material.

In some implementations of a method, such as any of those described in any of the preceding paragraphs of this summary, the second array of rings is located between the second sub-layer of filter material and the plurality of imaging regions.

In some implementations of a method, such as any of those described in any of the preceding paragraphs of this summary, the rings of the first array of rings define openings. The openings of the rings of the first array of rings each have a first diameter. The rings of the second array of rings define openings. The openings of the rings of the second array of rings each have a second diameter. The first diameter is different from the second diameter.

In some implementations of a method, such as any of those described in any of the preceding paragraphs of this summary, the first diameter is smaller than the second diameter.

In some implementations of a method, such as any of those described in any of the preceding paragraphs of this summary, the first diameter is approximately 700 nm.

In some implementations of a method, such as any of those described in any of the preceding paragraphs of this summary, the second diameter is approximately 900 nm.

In some implementations of a method, such as any of those described in any of the preceding paragraphs of this summary, the optical filter layer includes ferric oxide.

Another implementation relates to an apparatus that includes a flow cell body defining a channel to receive fluid, the channel having a floor extending along a length of the flow cell body. The apparatus further includes a plurality of wells positioned along the floor of the channel, the plurality of wells forming an array along a length of the floor of the channel. The apparatus further includes an optical filter layer positioned under the floor of the channel, the optical filter including at least a portion spanning uninterruptedly along a length corresponding to the length of the array of wells. The apparatus further includes a plurality of imaging regions positioned under the optical filter layer, each imaging region of the plurality of imaging regions being positioned directly under at least one corresponding well of the plurality of wells, such that each well and corresponding imaging region cooperate to form a sensing relationship. The optical filter layer is configured to permit one or more selected wavelengths of light to pass from each well to the imaging region forming a sensing relationship with the well. The optical filter layer is configured to reduce transmission of excitation light directed toward the plurality of wells, the optical filter layer being further configured to reduce transmission of light emitted from each well to imaging regions not forming a sensing relationship with the well.

In some implementations of an apparatus, such as that described in the preceding paragraph of this summary, the floor of the channel defines the plurality of wells.

In some implementations of an apparatus, such as any of those described in any of the preceding paragraphs of this summary, the wells include nanowells.

In some implementations of an apparatus, such as any of those described in any of the preceding paragraphs of this summary, the flow cell body defines a plurality of channels, the channels being oriented parallel with each other, each channel of the plurality of channels having a floor with a plurality of wells.

In some implementations of an apparatus, such as any of those described in any of the preceding paragraphs of this summary, the plurality of channels form an array along a width of the flow cell body, the optical layer including at least a portion spanning uninterruptedly along a width corresponding to the width of the array of channels.

In some implementations of an apparatus, such as any of those described in any of the preceding paragraphs of this summary, the apparatus further includes a plurality of imaging sensors, each imaging sensor forming a corresponding imaging region of the plurality of imaging regions.

In some implementations of an apparatus, such as any of those described in any of the preceding paragraphs of this summary, each imaging sensor includes a photodiode.

In some implementations of an apparatus, such as any of those described in any of the preceding paragraphs of this summary, the apparatus further includes an imaging chip, the imaging chip spanning along a length corresponding to the length of the array of wells, the imaging chip defining the plurality of imaging regions.

In some implementations of an apparatus, such as any of those described in any of the preceding paragraphs of this summary, the imaging sensor defines a plurality of photodiodes, each imaging region of the plurality of imaging regions being defined by one or more photodiodes of the plurality of photodiodes.

In some implementations of an apparatus, such as any of those described in any of the preceding paragraphs of this summary, the imaging chip includes a CMOS chip.

In some implementations of an apparatus, such as any of those described in any of the preceding paragraphs of this summary, the apparatus further includes a light source, the light source being configured to emit light at an excitation wavelength, the excitation wavelength being configured to cause one or more fluorophores in the wells to fluoresce at an emission wavelength.

In some implementations of an apparatus, such as any of those described in any of the preceding paragraphs of this summary, the optical filter layer is configured to substantially prevent transmission of light at the excitation wavelength to the plurality of imaging regions.

In some implementations of an apparatus, such as any of those described in any of the preceding paragraphs of this summary, the optical filter is configured to absorb light at the excitation wavelength.

In some implementations of an apparatus, such as any of those described in any of the preceding paragraphs of this summary, the optical filter layer is configured to absorb at least some light at the emission wavelength.

In some implementations of an apparatus, such as any of those described in any of the preceding paragraphs of this summary, the optical filter layer is configured to reduce transmission of light from each well to imaging regions not forming a sensing relationship with the well by inducing loss in light transmitted from the wells.

In some implementations of an apparatus, such as any of those described in any of the preceding paragraphs of this summary, the apparatus further includes a plurality of shields, each shield of the plurality of shields to block optical rays between a corresponding reaction site and an imaging region of the plurality of imaging regions that does not form a sensing pair with the corresponding reaction site.

In some implementations of an apparatus, such as any of those described in any of the preceding paragraphs of this summary, each shield of the plurality of shields being aligned with a corresponding sensing pair.

In some implementations of an apparatus, such as any of those described in any of the preceding paragraphs of this summary, the optical filter layer extends along a first height between the floor of the channel and the plurality of imaging regions, the plurality of shields extending along a second height between the floor of the channel and the plurality of imaging regions, the first height being greater than the second height such that the plurality of shields extend along only a portion of the first height.

In some implementations of an apparatus, such as any of those described in any of the preceding paragraphs of this summary, the plurality of shields extend from an underside of the floor, the plurality of shields having lower ends vertically terminating within the optical filter layer.

In some implementations of an apparatus, such as any of those described in any of the preceding paragraphs of this summary, the plurality of shields extend from an upper side of the plurality of imaging regions, the plurality of shields having upper ends vertically terminating within the optical filter layer.

In some implementations of an apparatus, such as any of those described in any of the preceding paragraphs of this summary, the optical filter layer is configured to permit transmission of light at wavelengths greater than approximately 600 nm.

In some implementations of an apparatus, such as any of those described in any of the preceding paragraphs of this summary, the optical filter layer is configured to substantially prevent transmission of light at wavelengths less than approximately 500 nm.

In some implementations of an apparatus, such as any of those described in any of the preceding paragraphs of this summary, the optical filter layer is configured to permit transmission of light at wavelengths greater than approximately 600 nm and prevent transmission of light at wavelengths less than approximately 500 nm.

In some implementations of an apparatus, such as any of those described in any of the preceding paragraphs of this summary, the optical filter layer is configured to absorb some light at wavelengths between approximately 500 nm and approximately 600 nm while permitting transmission of some light at wavelengths between approximately 500 nm and approximately 600 nm.

In some implementations of an apparatus, such as any of those described in any of the preceding paragraphs of this summary, the optical filter layer includes a combination of an orange dye and a black dye.

In some implementations of an apparatus, such as any of those described in any of the preceding paragraphs of this summary, the flow cell body includes a cover positioned over the channel.

In some implementations of an apparatus, such as any of those described in any of the preceding paragraphs of this summary, the cover includes glass.

In some implementations of an apparatus, such as any of those described in any of the preceding paragraphs of this summary, the floor includes glass.

In some implementations of an apparatus, such as any of those described in any of the preceding paragraphs of this summary, the imaging regions is integral with the flow cell body.

In some implementations of an apparatus, such as any of those described in any of the preceding paragraphs of this summary, the optical filter layer has a transmittance coefficient ranging from approximately 0.01 to approximately 0.5.

In some implementations of an apparatus, such as any of those described in any of the preceding paragraphs of this summary, the optical filter layer has a transmittance coefficient ranging from approximately 0.2 to approximately 0.4.

In some implementations of an apparatus, such as any of those described in any of the preceding paragraphs of this summary, the optical filter layer and floor cooperate to define a height dimension, the height dimension corresponding to a distance between a top of the floor and a bottom of the optical filter layer. The plurality of wells define a pitch dimension, the pitch dimension corresponding to a distance between a center of one well of the plurality of wells to a center of an adjacent well of the plurality of wells. The height dimension and pitch dimension provide a height-to-pitch ratio ranging from approximately 3 to approximately 5.

In some implementations of an apparatus, such as any of those described in any of the preceding paragraphs of this summary, the height dimension and pitch dimension providing a height-to-pitch ratio of approximately 4.

In some implementations of an apparatus, such as any of those described in any of the preceding paragraphs of this summary, the apparatus lacks any shields between the plurality of wells and the plurality of imaging regions.

In some implementations of an apparatus, such as any of those described in any of the preceding paragraphs of this summary, the optical filter layer has a thickness ranging from approximately 200 nm to approximately 5 μm.

In some implementations of an apparatus, such as any of those described in any of the preceding paragraphs of this summary, the optical filter layer has a thickness of approximately 1 μm.

In some implementations of an apparatus, such as any of those described in any of the preceding paragraphs of this summary, the optical filter layer is separated from each well by a distance ranging from approximately 25 nm to approximately 500 nm.

In some implementations of an apparatus, such as any of those described in any of the preceding paragraphs of this summary, the apparatus further includes a passivation layer interposed between the optical filter layer and the plurality of imaging regions.

In some implementations of an apparatus, such as any of those described in any of the preceding paragraphs of this summary, the passivation layer includes silicon dioxide.

In some implementations of an apparatus, such as any of those described in any of the preceding paragraphs of this summary, the passivation layer having a thickness ranging from approximately 10 nm to approximately 200 nm.

In some implementations of an apparatus, such as any of those described in any of the preceding paragraphs of this summary, the imaging regions are separated from each other by a pitch distance ranging from approximately 0.5 μm to approximately 25 μm.

In some implementations of an apparatus, such as any of those described in any of the preceding paragraphs of this summary, the imaging regions are separated from each other by a pitch distance of approximately 1 μm.

In some implementations of an apparatus, such as any of those described in any of the preceding paragraphs of this summary, the imaging regions are separated from each other by a pitch distance of approximately 2 μm.

In some implementations of an apparatus, such as any of those described in any of the preceding paragraphs of this summary, the optical filter layer includes a first sub-layer of filter material and a second sub-layer of filter material.

In some implementations of an apparatus, such as any of those described in any of the preceding paragraphs of this summary, the first sub-layer of filter material and the second sub-layer of filter material have the same thickness.

In some implementations of an apparatus, such as any of those described in any of the preceding paragraphs of this summary, the apparatus further includes a plurality of rings, the plurality of rings being positioned adjacent to one or both of the first sub-layer of filter material or the second sub-layer of filter material.

In some implementations of an apparatus, such as any of those described in any of the preceding paragraphs of this summary, each ring of the plurality of rings is associated with a corresponding sensing pair of the sensing pairs formed by each well and corresponding imaging region.

In some implementations of an apparatus, such as any of those described in any of the preceding paragraphs of this summary, each ring of the plurality of rings is centered about an axis passing through a center of a well and imaging region of the sensing pair corresponding with the ring.

In some implementations of an apparatus, such as any of those described in any of the preceding paragraphs of this summary, each ring of the plurality of rings includes a metal.

In some implementations of an apparatus, such as any of those described in any of the preceding paragraphs of this summary, the metal includes tungsten or aluminum.

In some implementations of an apparatus, such as any of those described in any of the preceding paragraphs of this summary, each ring of the plurality of rings has a thickness ranging from approximately 25 nm to approximately 100 nm.

In some implementations of an apparatus, such as any of those described in any of the preceding paragraphs of this summary, the plurality of rings includes a first array of rings and a second array of rings. The first array of rings is located at a first vertical position between the wells and the plurality of imaging regions. The second array of rings is located at a second vertical position between the wells and the plurality of imaging regions.

In some implementations of an apparatus, such as any of those described in any of the preceding paragraphs of this summary, the first array of rings is located at an interface between the first sub-layer of filter material and the second sub-layer of filter material.

In some implementations of an apparatus, such as any of those described in any of the preceding paragraphs of this summary, the second array of rings is located between the second sub-layer of filter material and the plurality of imaging regions.

In some implementations of an apparatus, such as any of those described in any of the preceding paragraphs of this summary, the rings of the first array of rings define openings. The openings of the rings of the first array of rings each have a first diameter. The rings of the second array of rings define openings. The openings of the rings of the second array of rings each have a second diameter. The first diameter is different from the second diameter.

In some implementations of an apparatus, such as any of those described in any of the preceding paragraphs of this summary, the first diameter is smaller than the second diameter.

In some implementations of an apparatus, such as any of those described in any of the preceding paragraphs of this summary, the first diameter is approximately 700 nm.

In some implementations of an apparatus, such as any of those described in any of the preceding paragraphs of this summary, the second diameter is approximately 900 nm.

In some implementations of an apparatus, such as any of those described in any of the preceding paragraphs of this summary, the optical filter layer includes ferric oxide.

Another implementation relates to a method of manufacturing a flow cell. The method includes forming an optical filter layer over an imaging layer, the imaging layer defining a plurality of imaging regions, the imaging layer extending along a first length, the imaging layer being operable to capture images at the plurality of imaging regions. The optical filter layer extends continuously along the first length. The method further includes positioning a floor over the optical filter layer, the floor extending along the first length of the flow cell, the floor defining a plurality of reaction sites over the optical filter layer, the plurality of reaction sites forming an array along the first length such that the optical filter layer extends continuously along a region under all the reaction sites of the plurality of reaction sites, each reaction site of the plurality of reaction sites being positioned directly over a corresponding imaging region of the plurality of imaging regions such that each reaction site cooperates with a corresponding imaging region to form a sensing relationship. The method further includes positioning a cover over the floor, the floor and the cover cooperating to define a fluid channel, the fluid channel extending along the first length. The cover, the floor, the optical filter layer, and the imaging layer cooperate to form at least a portion of a flow cell body. The optical filter layer is configured to permit one or more selected wavelengths of light to pass from each reaction site to the imaging region forming a sensing relationship with the reaction site. The optical filter layer is configured to reduce transmission of excitation light directed toward the plurality of reaction sites, the optical filter layer being further configured to reduce transmission of light emitted from each reaction site to imaging regions not forming a sensing relationship with the reaction site.

In some implementations of a method, such as that described in the preceding paragraph of this summary, the imaging layer comprising a CMOS chip.

In some implementations of a method, such as any of those described in any of the preceding paragraphs of this summary, the imaging regions include CMOS photodiodes of the CMOS chip.

In some implementations of a method, such as any of those described in any of the preceding paragraphs of this summary, the optical filter layer includes a combination of an orange dye and a black dye.

In some implementations of a method, such as any of those described in any of the preceding paragraphs of this summary, the floor includes glass.

In some implementations of a method, such as any of those described in any of the preceding paragraphs of this summary, the floor defines a plurality of nanowells. The plurality of nanowells define the plurality of reaction sites.

In some implementations of a method, such as any of those described in any of the preceding paragraphs of this summary, the cover includes glass.

In some implementations of a method, such as any of those described in any of the preceding paragraphs of this summary, the flow cell body has a second length, the second length being greater than the first length.

In some implementations of a method, such as any of those described in any of the preceding paragraphs of this summary, the fluid channel defines a width, the plurality of reaction sites further forming an array across the width of the fluid channel.

In some implementations of a method, such as any of those described in any of the preceding paragraphs of this summary, the optical filter layer extends continuously across the width of the fluid channel.

In some implementations of a method, such as any of those described in any of the preceding paragraphs of this summary, the floor and the cover cooperate to define a plurality of fluid channels, the fluid channels being oriented parallel with each other, the plurality of fluid channels forming an array across a width of the flow cell body.

In some implementations of a method, such as any of those described in any of the preceding paragraphs of this summary, each fluid channel of the plurality of fluid channels contains a corresponding set of reaction sites of the plurality of reaction sites.

In some implementations of a method, such as any of those described in any of the preceding paragraphs of this summary, the optical filter layer extends continuously across the width of the flow cell body.

In some implementations of a method, such as any of those described in any of the preceding paragraphs of this summary, the optical filter layer is configured to reduce transmission of light from each reaction site to imaging regions not forming a sensing relationship with the reaction site by inducing loss in light transmitted from the reaction sites.

In some implementations of a method, such as any of those described in any of the preceding paragraphs of this summary, the method further includes forming a plurality of shields within the optical filter layer, each shield of the plurality of shields to block optical rays between a corresponding reaction site and an imaging region of the plurality of imaging regions that does not form a sensing pair with the corresponding reaction site.

In some implementations of a method, such as any of those described in any of the preceding paragraphs of this summary, each shield of the plurality of shields being aligned with a corresponding sensing pair.

In some implementations of a method, such as any of those described in any of the preceding paragraphs of this summary, the optical filter layer extends along a first height between the floor and the imaging layer, the plurality of shields extending along a second height between the floor and the imaging layer, the first height being greater than the second height such that the plurality of shields extend along only a portion of the first height.

In some implementations of a method, such as any of those described in any of the preceding paragraphs of this summary, the plurality of shields extend from an underside of the floor, the plurality of shields having lower ends vertically terminating within the optical filter layer such that a region of the optical layer extends between the lower ends and the imaging layer.

In some implementations of a method, such as any of those described in any of the preceding paragraphs of this summary, the plurality of shields extend from an upper side of the imaging layer, the plurality of shields having upper ends vertically terminating within the optical filter layer such that a region of the optical layer extends between the upper ends and the floor.

In some implementations of a method, such as any of those described in any of the preceding paragraphs of this summary, the optical filter layer is configured to permit transmission of light at wavelengths greater than approximately 600 nm.

In some implementations of a method, such as any of those described in any of the preceding paragraphs of this summary, the optical filter layer is configured to substantially prevent transmission of light at wavelengths less than approximately 500 nm.

In some implementations of a method, such as any of those described in any of the preceding paragraphs of this summary, the optical filter layer is configured to permit transmission of light at wavelengths greater than approximately 600 nm and prevent transmission of light at wavelengths less than approximately 500 nm.

In some implementations of a method, such as any of those described in any of the preceding paragraphs of this summary, the optical filter layer is configured to absorb some light at wavelengths between approximately 500 nm and approximately 600 nm while permitting transmission of some light at wavelengths between approximately 500 nm and approximately 600 nm.

In some implementations of a method, such as any of those described in any of the preceding paragraphs of this summary, the optical filter layer has a transmittance coefficient ranging from approximately 0.01 to approximately 0.5.

In some implementations of a method, such as any of those described in any of the preceding paragraphs of this summary, the optical filter layer has a transmittance coefficient ranging from approximately 0.2 to approximately 0.4.

In some implementations of a method, such as any of those described in any of the preceding paragraphs of this summary, the optical filter layer and floor cooperate to define a height dimension, the height dimension corresponding to a distance between a top of the floor and a bottom of the optical filter layer. The plurality of reaction sites define a pitch dimension. The pitch dimension corresponds to a distance between a center of one reaction site of the plurality of reaction sites to a center of an adjacent reaction site of the plurality of reaction sites. The height dimension and pitch dimension provide a height-to-pitch ratio ranging from approximately 3 to approximately 5.

In some implementations of a method, such as any of those described in any of the preceding paragraphs of this summary, the height dimension and pitch dimension provide a height-to-pitch ratio of approximately 4.

In some implementations of a method, such as any of those described in any of the preceding paragraphs of this summary, the optical filter layer has a thickness ranging from approximately 200 nm to approximately 5 μm.

In some implementations of a method, such as any of those described in any of the preceding paragraphs of this summary, the optical filter layer has a thickness of approximately 1 μm.

In some implementations of a method, such as any of those described in any of the preceding paragraphs of this summary, the optical filter layer is separated from each reaction site by a distance ranging from approximately 25 nm to approximately 500 nm.

In some implementations of a method, such as any of those described in any of the preceding paragraphs of this summary, the method further includes providing a passivation layer interposed between the optical filter layer and the plurality of imaging regions.

In some implementations of a method, such as any of those described in any of the preceding paragraphs of this summary, the passivation layer includes silicon dioxide.

In some implementations of a method, such as any of those described in any of the preceding paragraphs of this summary, the passivation layer having a thickness ranging from approximately 10 nm to approximately 200 nm.

In some implementations of a method, such as any of those described in any of the preceding paragraphs of this summary, the imaging regions are separated from each other by a pitch distance ranging from approximately 0.5 μm to approximately 25 μm.

In some implementations of a method, such as any of those described in any of the preceding paragraphs of this summary, the imaging regions are separated from each other by a pitch distance of approximately 1 μm.

In some implementations of a method, such as any of those described in any of the preceding paragraphs of this summary, the imaging regions are separated from each other by a pitch distance of approximately 2 μm.

In some implementations of a method, such as any of those described in any of the preceding paragraphs of this summary, the optical filter layer includes a first sub-layer of filter material and a second sub-layer of filter material.

In some implementations of a method, such as any of those described in any of the preceding paragraphs of this summary, the first sub-layer of filter material and the second sub-layer of filter material have the same thickness.

In some implementations of a method, such as any of those described in any of the preceding paragraphs of this summary, the method further includes providing a plurality of rings, the plurality of rings being positioned adjacent to one or both of the first sub-layer of filter material or the second sub-layer of filter material.

In some implementations of a method, such as any of those described in any of the preceding paragraphs of this summary, each ring of the plurality of rings is associated with a corresponding sensing pair of the sensing pairs formed by each reaction site and corresponding imaging region.

In some implementations of a method, such as any of those described in any of the preceding paragraphs of this summary, each ring of the plurality of rings is centered about an axis passing through a center of a reaction site and imaging region of the sensing pair corresponding with the ring.

In some implementations of a method, such as any of those described in any of the preceding paragraphs of this summary, each ring of the plurality of rings includes a metal.

In some implementations of a method, such as any of those described in any of the preceding paragraphs of this summary, the metal includes tungsten or aluminum.

In some implementations of a method, such as any of those described in any of the preceding paragraphs of this summary, each ring of the plurality of rings has a thickness ranging from approximately 25 nm to approximately 100 nm.

In some implementations of a method, such as any of those described in any of the preceding paragraphs of this summary, the plurality of rings includes a first array of rings and a second array of rings. The first array of rings is located at a first vertical position between the reaction sites and the plurality of imaging regions. The second array of rings is located at a second vertical position between the reaction sites and the plurality of imaging regions.

In some implementations of a method, such as any of those described in any of the preceding paragraphs of this summary, the first array of rings is located at an interface between the first sub-layer of filter material and the second sub-layer of filter material.

In some implementations of a method, such as any of those described in any of the preceding paragraphs of this summary, the second array of rings is located between the second sub-layer of filter material and the plurality of imaging regions.

In some implementations of a method, such as any of those described in any of the preceding paragraphs of this summary, the rings of the first array of rings define openings. The openings of the rings of the first array of rings each have a first diameter. The rings of the second array of rings define openings. The openings of the rings of the second array of rings each have a second diameter. The first diameter is different from the second diameter.

In some implementations of a method, such as any of those described in any of the preceding paragraphs of this summary, the first diameter is smaller than the second diameter.

In some implementations of a method, such as any of those described in any of the preceding paragraphs of this summary, the first diameter is approximately 700 nm.

In some implementations of a method, such as any of those described in any of the preceding paragraphs of this summary, the second diameter is approximately 900 nm.

In some implementations of a method, such as any of those described in any of the preceding paragraphs of this summary, the optical filter layer includes ferric oxide.

While multiple examples are described, still other examples of the described subject matter will become apparent to those skilled in the art from the following detailed description and drawings, which show and describe illustrative examples of disclosed subject matter. As will be realized, the disclosed subject matter is capable of modifications in various aspects, all without departing from the spirit and scope of the described subject matter. Accordingly, the drawings and detailed description are to be regarded as illustrative in nature and not restrictive.

### BRIEF DESCRIPTION OF THE DRAWINGS

FIG. 1 depicts a block diagram of an example of a system for biological or chemical analysis.

FIG. 2 depicts a block diagram of an example of a system controller that may be used in the system of FIG. 1.

FIG. 3 depicts a cross-sectional view of an example of a biosensor that may be used in the system of FIG. 1.

FIG. 4 depicts a cross-sectional view of an enlarged portion of the biosensor of FIG. 3.

FIG. 5 depicts a cross-sectional view of another example of a biosensor that may be used in the system of FIG. 1.

FIG. 6 depicts a cross-sectional view of another example of a biosensor that may be used in the system of FIG. 1.

FIG. 7 depicts a graph plotting optical characteristics associated with the biosensor of FIG. 6.

FIG. 8 depicts a graph plotting point spread function data associated with different versions of the biosensor of FIG. 6.

FIG. 9 depicts an example of an image captured using one version of the biosensor of FIG. 6.

FIG. 10 depicts an example of an image captured using another version of the biosensor of FIG. 6.

FIG. 11 depicts an example of an image captured using another version of the biosensor of FIG. 6.

FIG. 12 depicts a cross-sectional view of another example of a biosensor that may be used in the system of FIG. 1.

FIG. 13 depicts a graph plotting optical characteristics associated with the biosensor of FIG. 12.

FIG. 14 depicts a graph plotting point spread function data associated with different versions of the biosensor of FIG. 12.

FIG. 15 depicts an example of an image captured using a version of the biosensor of FIG. 12.

FIG. 16 depicts an example of an image captured using another version of the biosensor of FIG. 12.

FIG. 17 depicts an example of an image captured using a version of the biosensor of FIG. 12, with a reference box shown on the image.

FIG. 18 depicts a graph plotting examples of power spread over pixels in different versions of the biosensor of FIG. 12.

FIG. 19 depicts a cross-sectional view of another example of a biosensor that may be used in the system of FIG. 1.

FIG. 20 depicts a cross-sectional view of another example of a biosensor that may be used in the system of FIG. 1.

FIG. 21 depicts a cross-sectional view of another example of a biosensor that may be used in the system of FIG. 1.

FIG. 22 depicts a cross-sectional view of another example of a biosensor that may be used in the system of FIG. 1.

### DETAILED DESCRIPTION

I. Overview of System for Biological or Chemical Analysis

Examples described herein may be used in various biological or chemical processes and systems for academic or commercial analysis. More specifically, examples described herein may be used in various processes and systems where it is desired to detect an event, property, quality, or characteristic that is indicative of a designated reaction. For instance, examples described herein include cartridges, biosensors, and their components as well as bioassay systems that operate with cartridges and biosensors. In particular examples, the cartridges and biosensors include a flow cell and one or more image sensors that are coupled together in a substantially unitary structure.

The bioassay systems may be configured to perform a plurality of designated reactions that may be detected individually or collectively. The biosensors and bioassay systems may be configured to perform numerous cycles in which the plurality of designated reactions occurs in parallel. For example, the bioassay systems may be used to sequence a dense array of DNA features through iterative cycles of enzymatic manipulation and image acquisition. The cartridges and biosensors may include one or more microfluidic channels that deliver reagents or other reaction components to a well or reaction site. In some examples, the wells or reaction sites are randomly distributed across a substantially planar surface. For example, the wells or reaction sites may have an uneven distribution in which some wells or reaction sites are located closer to each other than other wells or reaction sites. In other examples, the wells or reaction sites are patterned across a substantially planar surface in a predetermined manner. Each of the wells or reaction sites may be associated with one or more image sensors that detect light from the associated reaction site. Yet in other examples, the wells or reaction sites are located in reaction chambers that compartmentalize the designated reactions therein.

In some examples, image sensors may detect light emitted from wells or reaction sites and the signals indicating photons emitted from the wells or reaction sites and detected by the individual image sensors may be referred to as those sensors' illumination values. These illumination values may be combined into an image indicating photons as detected from the wells or reaction sites. Such an image may be referred to as a raw image. Similarly, when an image is composed of values which have been processed, such as to computationally correct for crosstalk, rather than being composed of the values directly detected by individual image sensors, that image may be referred to as a sharpened image.

The following detailed description of certain examples will be better understood when read in conjunction with the appended drawings. To the extent that the figures illustrate diagrams of the functional blocks of various examples, the functional blocks are not necessarily indicative of the division between hardware components. Thus, for example, one or more of the functional blocks (e.g., processors or memories) may be implemented in a single piece of hardware (e.g., a general-purpose signal processor or random-access memory, hard disk, or the like). Similarly, the programs may be stand-alone programs, may be incorporated as subroutines in an operating system, may be functions in an installed software package, and the like. It should be understood that the various examples are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one example” are not intended to be interpreted as excluding the existence of additional examples that also incorporate the recited features. Moreover, unless explicitly stated to the contrary, examples “comprising” or “having” an element or a plurality of elements having a particular property may include additional elements whether or not they have that property.

As used herein, a “designated reaction” includes a change in at least one of a chemical, electrical, physical, or optical property (or quality) of an analyte-of-interest. In some examples, the designated reaction is a positive binding event (e.g., incorporation of a fluorescently labeled biomolecule with the analyte-of-interest). More generally, the designated reaction may be a chemical transformation, chemical change, or chemical interaction. In some examples, the designated reaction includes the incorporation of a fluorescently-labeled molecule to an analyte. The analyte may be an oligonucleotide and the fluorescently-labeled molecule may be a nucleotide. The designated reaction may be detected when an excitation light is directed toward the oligonucleotide having the labeled nucleotide, and the fluorophore emits a detectable fluorescent signal. In alternative examples, the detected fluorescence is a result of chemiluminescence or bioluminescence. A designated reaction may also increase fluorescence (or Förster) resonance energy transfer (FRET), for example, by bringing a donor fluorophore in proximity to an acceptor fluorophore, decrease FRET by separating donor and acceptor fluorophores, increase fluorescence by separating a quencher from a fluorophore or decrease fluorescence by co-locating a quencher and fluorophore.

As used herein, a “reaction component” or “reactant” includes any substance that may be used to obtain a designated reaction. For example, reaction components include reagents, enzymes, samples, other biomolecules, and buffer solutions. The reaction components may be delivered to a reaction site in a solution and/or immobilized at a reaction site. The reaction components may interact directly or indirectly with another substance, such as the analyte-of-interest.

As used herein, the term “reaction site” is a localized region where a designated reaction may occur. A reaction site may include support surfaces of a substrate where a substance may be immobilized thereon. For example, a reaction site may include a substantially planar surface in a channel of a flow cell that has a colony of nucleic acids thereon. The nucleic acids in the colony may have the same sequence, being for example, clonal copies of a single stranded or double stranded template. However, in some examples a reaction site may contain only a single nucleic acid molecule, for example, in a single stranded or double stranded form. Furthermore, a plurality of wells or reaction sites may be randomly distributed along the support surface or arranged in a predetermined manner (e.g., side-by-side in a matrix, such as in microarrays). A reaction site may also include a reaction chamber that at least partially defines a spatial region or volume configured to compartmentalize the designated reaction. As used herein, the term “reaction chamber” includes a spatial region that is in fluid communication with a flow channel. The reaction chamber may be at least partially separated from the surrounding environment or other spatial regions. For example, a plurality of reaction chambers may be separated from each other by shared walls. As a more specific example, the reaction chamber may include a cavity defined by interior surfaces of a well and have an opening or aperture so that the cavity may be in fluid communication with a flow channel. Examples of biosensors including such reaction chambers are described in greater detail in U.S. Pat. No. 9,9096,899, entitled “Microdevices and Biosensor Cartridges for Biological or Chemical Analysis and Systems and Methods for the Same,” issued Aug. 4, 2015, the disclosure of which is incorporated herein by reference, in its entirety.

In some examples, the reaction chambers are sized and shaped relative to solids (including semi-solids) so that the solids may be inserted, fully or partially, therein. For example, the reaction chamber may be sized and shaped to accommodate only one capture bead. The capture bead may have clonally amplified DNA or other substances thereon. Alternatively, the reaction chamber may be sized and shaped to receive an approximate number of beads or solid substrates. As another example, the reaction chambers may also be filled with a porous gel or substance that is configured to control diffusion or filter fluids that may flow into the reaction chamber.

In some examples, image sensors (e.g., photodiodes) are associated with corresponding wells or reaction sites. An image sensor that is associated with a reaction site is configured to detect light emissions from the associated reaction site when a designated reaction has occurred at the associated reaction site. In some cases, a plurality of image sensors (e.g., several pixels of a camera device) may be associated with a single reaction site. In other cases, a single image sensor (e.g., a single pixel) may be associated with a single reaction site or with a group of wells or reaction sites. The image sensor, the reaction site, and other features of the biosensor may be configured so that at least some of the light is directly detected by the image sensor without being reflected.

As used herein, the term “adjacent” when used with respect to two wells or reaction sites means no other reaction site is located between the two wells or reaction sites. The term “adjacent” may have a similar meaning when used with respect to adjacent detection paths and adjacent image sensors (e.g., adjacent image sensors have no other image sensor therebetween). In some cases, a reaction site may not be adjacent to another reaction site; but may still be within an immediate vicinity of the other reaction site. A first reaction site may be in the immediate vicinity of a second reaction site when fluorescent emission signals from the first reaction site are detected by the image sensor associated with the second reaction site. More specifically, a first reaction site may be in the immediate vicinity of a second reaction site when the image sensor associated with the second reaction site detects, for example, crosstalk from the first reaction site. Adjacent wells or reaction sites may be contiguous, such that they abut each other, or the adjacent sites may be non-contiguous, having an intervening space between.

As used herein, a “substance” includes items or solids, such as capture beads, as well as biological or chemical substances. As used herein, a “biological or chemical substance” includes biomolecules, samples-of-interest, analytes-of-interest, and other chemical compound(s). A biological or chemical substance may be used to detect, identify, or analyze other chemical compound(s), or function as intermediaries to study or analyze other chemical compound(s). In particular examples, the biological or chemical substances include a biomolecule. As used herein, a “biomolecule” includes at least one of a biopolymer, nucleoside, nucleic acid, polynucleotide, oligonucleotide, protein, enzyme, polypeptide, antibody, antigen, ligand, receptor, polysaccharide, carbohydrate, polyphosphate, cell, tissue, organism, or fragment thereof or any other biologically active chemical compound(s) such as analogs or mimetics of the aforementioned species.

Biomolecules, samples, and biological or chemical substances may be naturally occurring or synthetic and may be suspended in a solution or mixture within a spatial region. Biomolecules, samples, and biological or chemical substances may also be bound to a solid phase or gel material. Biomolecules, samples, and biological or chemical substances may also include a pharmaceutical composition. In some cases, biomolecules, samples, and biological or chemical substances of interest may be referred to as targets, probes, or analytes.

As used herein, a “biosensor” includes a structure having a plurality of wells or reaction sites that is configured to detect designated reactions that occur at or proximate to the wells or reaction sites. A biosensor may include a solid-state imaging device (e.g., CCD or CMOS imager) and, optionally, a flow cell mounted thereto. The flow cell may include at least one flow channel that is in fluid communication with the wells or reaction sites. As one specific example, the biosensor is configured to fluidically and electrically couple to a bioassay system. The bioassay system may deliver reactants to the wells or reaction sites according to a predetermined protocol (e.g., sequencing-by-synthesis) and perform a plurality of imaging events. For example, the bioassay system may direct solutions to flow along the wells or reaction sites. At least one of the solutions may include four types of nucleotides having the same or different fluorescent labels. The nucleotides may bind to corresponding oligonucleotides located at the wells or reaction sites. The bioassay system may then illuminate the wells or reaction sites using an excitation light source (e.g., solid-state light sources, such as light-emitting diodes or LEDs). The excitation light may have a predetermined wavelength or wavelengths, including a range of wavelengths. The excited fluorescent labels provide emission signals that may be detected by the image sensors.

As used herein, a “cartridge” includes a structure that is configured to hold a biosensor. In some examples, the cartridge may include additional features, such as the light source (e.g., LEDs) that are configured to provide excitation light to the wells or reaction sites of the biosensor. The cartridge may also include a fluidic storage system (e.g., storage for reagents, sample, and buffer) and a fluidic control system (e.g., pumps, valves, and the like) for fluidically transporting reaction components, sample, and the like to the wells or reaction sites. For example, after the biosensor is prepared or manufactured, the biosensor may be coupled to a housing or container of the cartridge. In some examples, the biosensors and the cartridges may be self-contained, disposable units. However, other examples may include an assembly with removable parts that allow a user to access an interior of the biosensor or cartridge for maintenance or replacement of components or samples. The biosensor and the cartridge may be removably coupled or engaged to larger bioassay systems, such as a sequencing system, that conducts controlled reactions therein.

As used herein, when the terms “removably” and “coupled” (or “engaged”) are used together to describe a relationship between the biosensor (or cartridge) and a system receptacle or interface of a bioassay system, the term is intended to mean that a connection between the biosensor (or cartridge) and the system receptacle is readily separable without destroying or damaging the system receptacle and/or the biosensor (or cartridge). Components are readily separable when the components may be separated from each other without undue effort or a significant amount of time spent in separating the components. For example, the biosensor (or cartridge) may be removably coupled or engaged to the system receptacle in an electrical manner such that the mating contacts of the bioassay system are not destroyed or damaged. The biosensor (or cartridge) may also be removably coupled or engaged to the system receptacle in a mechanical manner such that the features that hold the biosensor (or cartridge) are not destroyed or damaged. The biosensor (or cartridge) may also be removably coupled or engaged to the system receptacle in a fluidic manner such that the ports of the system receptacle are not destroyed or damaged. The system receptacle or a component is not considered to be destroyed or damaged if, for example, only a simple adjustment to the component (e.g., realignment) or a simple replacement (e.g., replacing a nozzle) is required.

As used herein, the term “fluid communication” or “fluidically coupled” refers to two spatial regions being connected together such that a liquid or gas may flow between the two spatial regions. For example, a microfluidic channel may be in fluid communication with a reaction chamber such that a fluid may flow freely into the reaction chamber from the microfluidic channel. The terms “in fluid communication” or “fluidically coupled” allow for two spatial regions being in fluid communication through one or more valves, restrictors, or other fluidic components to control or regulate a flow of fluid through a system.

The terms “substantially”, “approximately”, “about”, “relatively”, or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, fluctuations can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “immobilized,” when used with respect to a biomolecule or biological or chemical substance, includes substantially attaching the biomolecule or biological or chemical substance at a molecular level to a surface. For example, a biomolecule or biological or chemical substance may be immobilized to a surface of the substrate material using adsorption techniques including non-covalent interactions (e.g., electrostatic forces, van der Waals, and dehydration of hydrophobic interfaces) and covalent binding techniques where functional groups or linkers facilitate attaching the biomolecules to the surface. Immobilizing biomolecules or biological or chemical substances to a surface of a substrate material may be based upon the properties of the substrate surface, the liquid medium carrying the biomolecule or biological or chemical substance, and the properties of the biomolecules or biological or chemical substances themselves. In some cases, a substrate surface may be functionalized (e.g., chemically or physically modified) to facilitate immobilizing the biomolecules (or biological or chemical substances) to the substrate surface. The substrate surface may be first modified to have functional groups bound to the surface. The functional groups may then bind to biomolecules or biological or chemical substances to immobilize them thereon.

In some examples, nucleic acids can be attached to a surface and amplified. Examples of such amplification are described in U.S. Pat. No. 7,741,463, entitled “Method of Preparing Libraries of Template Polynucleotides,” issued Jun. 22, 2010, the disclosure of which is incorporated by reference herein, in its entirety. In some cases, repeated rounds of extension (e.g., amplification) using an immobilized primer and primer in solution may provide multiple copies of the nucleic acid.

In particular examples, the assay protocols executed by the systems and methods described herein include the use of natural nucleotides and also enzymes that are configured to interact with the natural nucleotides. Natural nucleotides include, for example, ribonucleotides or deoxyribonucleotides. Natural nucleotides can be in the mono-, di-, or tri-phosphate form and can have a base selected from adenine (A), Thymine (T), uracil (U), guanine (G) or cytosine (C). It will be understood however that non-natural nucleotides, modified nucleotides or analogs of the aforementioned nucleotides can be used.

In examples that include reaction chambers, items or solid substances (including semi-solid substances) may be disposed within the reaction chambers. When disposed, the item or solid may be physically held or immobilized within the reaction chamber through an interference fit, adhesion, or entrapment. Examples of items or solids that may be disposed within the reaction chambers include polymer beads, pellets, agarose gel, powders, quantum dots, or other solids that may be compressed and/or held within the reaction chamber. In some examples, a nucleic acid superstructure, such as a DNA ball, may be disposed in or at a reaction chamber, for example, by attachment to an interior surface of the reaction chamber or by residence in a liquid within the reaction chamber. A substance that is held or disposed in a reaction chamber can be in a solid, liquid, or gaseous state.

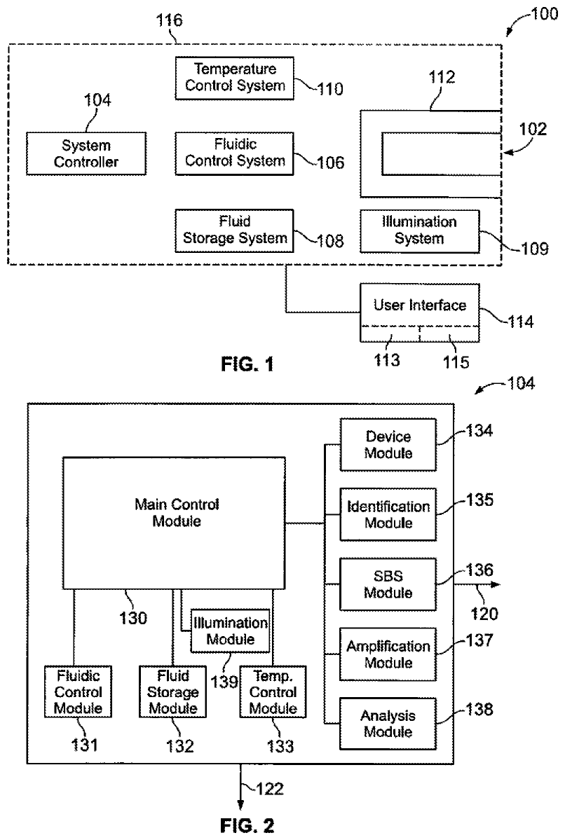

FIG. 1 is a block diagram of a bioassay system 100 for biological or chemical analysis formed in accordance with one example. The term “bioassay” is not intended to be limiting as the bioassay system 100 may operate to obtain any information or data that relates to at least one of a biological or chemical substance. In some examples, the bioassay system 100 is a workstation that may be similar to a bench-top device or desktop computer. For example, a majority (or all) of the systems and components for conducting the designated reactions may be within a common housing 116.

In particular examples, the bioassay system 100 is a nucleic acid sequencing system (or sequencer) configured for various applications, including but not limited to de novo sequencing, resequencing of whole genomes or target genomic regions, and metagenomics. The sequencer may also be used for DNA or RNA analysis. In some versions, the bioassay system 100 may also be configured to generate reaction sites in a biosensor. For example, the bioassay system 100 may be configured to receive a sample and generate surface attached clusters of clonally amplified nucleic acids derived from the sample. Each cluster may constitute or be part of a reaction site in the biosensor.

The bioassay system 100 may include a system receptacle or interface 112 that is configured to interact with a biosensor 102 to perform designated reactions within the biosensor 102. In the following description with respect to FIG. 1, the biosensor 102 is loaded into the system receptacle 112. However, it is understood that a cartridge that includes the biosensor 102 may be inserted into the system receptacle 112 and in some states the cartridge may be removed temporarily or permanently. As described above, the cartridge may include, among other things, fluidic control and fluidic storage components.

In particular examples, the bioassay system 100 is to perform a large number of parallel reactions within the biosensor 102. The biosensor 102 includes one or more wells or reaction sites where designated reactions may occur. The reaction sites may be, for example, immobilized to a solid surface of the biosensor or immobilized to beads (or other movable substrates) that are located within corresponding reaction chambers or wells of the biosensor. The reaction sites may include, for example, clusters of clonally amplified nucleic acids. The biosensor 102 may include a solid-state imaging device (e.g., CCD or CMOS imager) and a flow cell mounted thereto. The flow cell may include one or more flow channels that receive a solution from the bioassay system 100 and direct the solution toward the wells or reaction sites. Optionally, the biosensor 102 may engage a thermal element for transferring thermal energy into or out of the flow channel.

The bioassay system 100 may include various components, assemblies, and systems (or sub-systems) that interact with each other to perform a predetermined method or assay protocol for biological or chemical analysis. For example, the bioassay system 100 includes a system controller 104 that may communicate with the various components, assemblies, and sub-systems of the bioassay system 100 and also the biosensor 102. In addition to the system receptacle 112, the bioassay system 100 may also include a fluidic control system 106 to control the flow of fluid throughout a fluid network of the bioassay system 100 and the biosensor 102; a fluid storage system 108 that is to hold fluids (e.g., gas or liquids) that may be used by the bioassay system; a temperature control system 110 that may regulate the temperature of the fluid in the fluid network, the fluid storage system 108, and/or the biosensor 102; and an illumination system 111 that is to illuminate the biosensor 102. As described above, if a cartridge having the biosensor 102 is loaded into the system receptacle 112, the cartridge may also include fluidic control and fluidic storage components.

Also shown, the bioassay system 100 may include a user interface 114 that interacts with the user. For example, the user interface 114 may include a display 113 to display or request information from a user and a user input device 115 to receive user inputs. The bioassay system 100 may communicate with various components, including the biosensor 102 (e.g., in the form of a cartridge), to perform the designated reactions. The bioassay system 100 may also analyze data obtained from the biosensor to provide a user with desired information. The system controller 104 may include any processor-based or microprocessor-based system, including systems using microcontrollers, reduced instruction set computers (RISC), application specific integrated circuits (ASICs), field programmable gate array (FPGAs), logic circuits, and any other circuit or processor capable of executing functions described herein. The above examples are not intended to limit in any way the definition and/or meaning of the term system controller. In an example, the system controller 104 executes a set of instructions that are stored in one or more storage elements, memories, or modules in order to at least one of obtain and analyze detection data. Storage elements may be in the form of information sources or physical memory elements within the bioassay system 100.

The set of instructions may include various commands that instruct the bioassay system 100 or biosensor 102 to perform specific operations such as the methods and processes of the various examples described herein. The set of instructions may be in the form of a software program, which may form part of a tangible, non-transitory computer readable medium or media. As used herein, the terms “software” and “firmware” are interchangeable; and include any computer program stored in memory for execution by a computer, including RAM memory, ROM memory, EPROM memory, EEPROM memory, and non-volatile RAM (NVRAM) memory. The above memory types are only examples and are thus not limiting as to the types of memory usable for storage of a computer program.

The software may be in various forms such as system software or application software. Further, the software may be in the form of a collection of separate programs, or a program module within a larger program or a portion of a program module. The software also may include modular programming in the form of object-oriented programming. After obtaining the detection data, the detection data may be automatically processed by the bioassay system 100, processed in response to user inputs, or processed in response to a request made by another processing machine (e.g., a remote request through a communication link).

The system controller 104 may be connected to the biosensor 102 and the other components of the bioassay system 100 via communication links. The system controller 104 may also be communicatively connected to off-site systems or servers. The communication links may be hardwired or wireless. The system controller 104 may receive user inputs or commands, from the user interface 114 and the user input device 115.

The fluidic control system 106 includes a fluid network and is to direct and regulate the flow of one or more fluids through the fluid network. The fluid network may be in fluid communication with the biosensor 102 and the fluid storage system 108. For example, select fluids may be drawn from the fluid storage system 108 and directed to the biosensor 102 in a controlled manner; or the fluids may be drawn from the biosensor 102 and directed toward, for example, a waste reservoir in the fluid storage system 108.

The temperature control system 110 is to regulate the temperature of fluids at different regions of the fluid network, the fluid storage system 108, and/or the biosensor 102. For example, the temperature control system 110 may include a thermocycler that interfaces with the biosensor 102 and controls the temperature of the fluid that flows along the wells or reaction sites in the biosensor 102. The temperature control system 110 may also regulate the temperature of solid elements or components of the bioassay system 100 or the biosensor 102.

The fluid storage system 108 is in fluid communication with the biosensor 102 and may store various reaction components or reactants that are used to conduct the designated reactions therein. The fluid storage system 108 may also store fluids for washing or cleaning the fluid network and biosensor 102 and for diluting the reactants. For example, the fluid storage system 108 may include various reservoirs to store samples, reagents, enzymes, other biomolecules, buffer solutions, aqueous, and non-polar solutions, and the like. Furthermore, the fluid storage system 108 may also include waste reservoirs for receiving waste products from the biosensor 102.

The illumination system 111 may include a light source (e.g., one or more LEDs) and a plurality of optical components to illuminate the biosensor. Examples of light sources may include lasers, arc lamps, LEDs, or laser diodes. The optical components may include, for example, reflectors, dichroics, beam splitters, collimators, lenses, filters, wedges, prisms, mirrors, detectors, and the like. In versions that use an illumination system, the illumination system 111 may be configured to direct an excitation light to wells or reaction sites.

The system receptacle or interface 112 is to engage the biosensor 102 in at least one of a mechanical, electrical, or fluidic manner. The system receptacle 112 may hold the biosensor 102 in a desired orientation to facilitate the flow of fluid through the biosensor 102. The system receptacle 112 may also include electrical contacts that are to engage the biosensor 102 so that the bioassay system 100 may communicate with the biosensor 102 and/or provide power to the biosensor 102. Furthermore, the system receptacle 112 may include fluidic ports (e.g., nozzles) that are to engage the biosensor 102. In some examples, the biosensor 102 is removably coupled to the system receptacle 112 in a mechanical manner, in an electrical manner, and also in a fluidic manner.

FIG. 2 is a block diagram of the system controller 104 in an example. In one example, the system controller 104 includes one or more processors or modules that may communicate with one another. Each of the processors or modules may include an algorithm (e.g., instructions stored on a tangible and/or non-transitory computer readable storage medium) or sub-algorithms to perform particular processes. The system controller 104 is illustrated conceptually as a collection of modules, but may be implemented utilizing any combination of dedicated hardware boards, DSPs, processors, etc. Alternatively, the system controller 104 may be implemented utilizing an off-the-shelf PC with a single processor or multiple processors, with the functional operations distributed between the processors. As a further option, the modules described below may be implemented utilizing a hybrid configuration in which certain modular functions are performed utilizing dedicated hardware, while the remaining modular functions are performed utilizing an off-the-shelf PC and the like. The modules also may be implemented as software modules within a processing unit.

During operation, a communication link 120 may transmit information (e.g., commands) to or receive information (e.g., data) from the biosensor 102 (FIG. 1) and/or the sub-systems 106, 108, 110 (FIG. 1). A communication link 122 may receive user input from the user interface 114 (FIG. 1) and transmit data or information to the user interface 114. Data from the biosensor 102 or sub-systems 106, 108, 110 may be processed by the system controller 104 in real-time during a bioassay session. Additionally, or alternatively, data may be stored temporarily in a system memory during a bioassay session and processed in slower than real-time or off-line operation.

As shown in FIG. 2, the system controller 104 may include a plurality of modules 131-139 that communicate with a main control module 130. The main control module 130 may communicate with the user interface 114 (FIG. 1). Although the modules 131-139 are shown as communicating directly with the main control module 130, the modules 131-139 may also communicate directly with each other, the user interface 114, and the biosensor 102. Also, the modules 131-139 may communicate with the main control module 130 through the other modules.

The plurality of modules 131-139 include system modules 131-133, 139 that communicate with the sub-systems 106, 108, 110, and 111, respectively. The fluidic control module 131 may communicate with the fluidic control system 106 to control the valves and flow sensors of the fluid network for controlling the flow of one or more fluids through the fluid network. The fluid storage module 132 may notify the user when fluids are low or when the waste reservoir is at or near capacity. The fluid storage module 132 may also communicate with the temperature control module 133 so that the fluids may be stored at a desired temperature. The illumination module 139 may communicate with the illumination system 109 to illuminate the wells or reaction sites at designated times during a protocol, such as after the designated reactions (e.g., binding events) have occurred.

The plurality of modules 131-139 may also include a device module 134 that communicates with the biosensor 102 and an identification module 135 that determines identification information relating to the biosensor 102. The device module 134 may, for example, communicate with the system receptacle 112 to confirm that the biosensor has established an electrical and fluidic connection with the bioassay system 100. The identification module 135 may receive signals that identify the biosensor 102. The identification module 135 may use the identity of the biosensor 102 to provide other information to the user. For example, the identification module 135 may determine and then display a lot number, a date of manufacture, or a protocol that is recommended to be run with the biosensor 102.

The plurality of modules 131-139 may also include a detection data analysis module 138 that receives and analyzes the signal data (e.g., image data) from the biosensor 102. The signal data may be stored for subsequent analysis or may be transmitted to the user interface 114 to display desired information to the user. In some versions, the signal data may be processed by the solid-state imager (e.g., CMOS image sensor) before the detection data analysis module 138 receives the signal data.

Protocol modules 136 and 137 communicate with the main control module 130 to control the operation of the sub-systems 106, 108, and 110 when conducting predetermined assay protocols. The protocol modules 136 and 137 may include sets of instructions for instructing the bioassay system 100 to perform specific operations pursuant to predetermined protocols. As shown, the protocol module may be a sequencing-by-synthesis (SBS) module 136 that is configured to issue various commands for performing SBS processes. The illumination system 111 may provide an excitation light to the wells or reaction sites during an SBS process and/or other processes.

The plurality of protocol modules may also include a sample-preparation (or generation) module 137 that is to issue commands to the fluidic control system 106 and the temperature control system 110 for amplifying a product within the biosensor 102. For example, the biosensor 102 may be engaged to the bioassay system 100. The amplification module 137 may issue instructions to the fluidic control system 106 to deliver necessary amplification components to reaction chambers within the biosensor 102. In other versions, the wells or reaction sites may already contain some components for amplification, such as the template DNA and/or primers. After delivering the amplification components to the reaction chambers, the amplification module 137 may instruct the temperature control system 110 to cycle through different temperature stages according to known amplification protocols. In some examples, the amplification and/or nucleotide incorporation is performed isothermally.

The SBS module 136 may issue commands to perform bridge PCR where clusters of clonal amplicons are formed on localized areas within a channel of a flow cell. After generating the amplicons through bridge PCR, the amplicons may be “linearized” to make single stranded template DNA, or sstDNA, and a sequencing primer may be hybridized to a universal sequence that flanks a region of interest. For example, a reversible terminator-based SBS method may be used as set forth above or as follows. In some examples, the amplification and SBS modules may operate in a single assay protocol where, for example, template nucleic acid is amplified and subsequently sequenced within the same cartridge.

II. Example of Biosensor with Flow Cell Interposed Between Light Source and Image Sensors

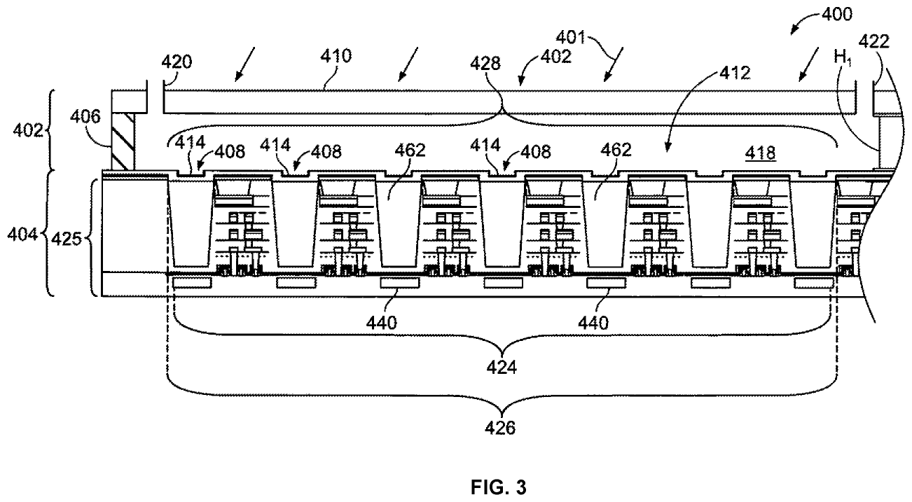

FIG. 3 illustrates a cross-section of a portion of an exemplary biosensor 400 formed in accordance with one example. The biosensor 400 may include similar features as the biosensor 102 (FIG. 1) described above and may be used in, for example, a cartridge as described herein. As shown, the biosensor 400 may include a flow cell 402 that is coupled directly or indirectly to a detection device 404. The flow cell 402 may be mounted to the detection device 404. In the present example, the flow cell 402 is affixed directly to the detection device 404 through one or more securing mechanisms (e.g., adhesive, bond, fasteners, and the like). In some examples, the flow cell 402 may be removably coupled to the detection device 404.

In the illustrated example, the detection device 404 includes a device base 425. In some versions, the device base 425 includes a plurality of stacked layers (e.g., silicon layer, dielectric layer, metal-dielectric layers, etc.). The device base 425 may include a sensor array 424 of image sensors 440, a guide array 426 of light guides 462, and a reaction array 428 of wells 408 that define reaction chambers having corresponding reaction sites 414. Since reaction sites 414 are defined in wells 408 in some versions, the terms “well” and “reaction site” may be used interchangeably herein. However, some variations may provide reaction sites atop elevated platforms or other structures that do not necessarily constitute wells 408 as shown in FIG. 3. The terms “well” and “reaction site” should therefore be read as including such alternative structures.

In certain examples, the components are arranged such that each image sensor 440 aligns with a single light guide 462 and a single reaction site 414. In such versions, a given image sensor 440 may be said to form a “sensing pair” with the reaction site 414 that is directly aligned with (e.g., positioned directly above) the image sensor 440. In versions where each image sensor 440 represents a single pixel, the image sensor 440 forming a sensing pair with a reaction site 414 may be referred to as the “center pixel” associated with that reaction site 414; while the image sensors 440 adjacent to the center pixel may be referred to as “neighbor pixels.” Similarly, an image sensor 440 that does not form a sensing pair with a given reaction site 414 may be referred to as a “neighbor sensor” with respect to that reaction site 414.

While just one reaction site 414 or well 408 defines a sensing pair with a given image sensor 440 or pixel in the present example, other variations may exist where a single image sensor 440 or pixel is positioned directly under two or more reaction sites 414 or wells 408. Examples of such variations are described in greater detail below. It should be understood that the term “sensing pair” may also refer to the relationships between such reaction sites 414 or wells 408 and the corresponding image sensors 440 or pixels. In other words, the term “sensing pair” should not be read as being limited only to structural arrangements where there is only a 1:1 pixel-to-reaction site ratio, pixel-to-well ratio, sensor-to-reaction site ratio, or sensor-to-well ratio. The term the term “sensing pair” may also apply to structural arrangements providing two or more wells 408 or reaction sites 414 per pixel or sensor 440. Such a sensing pair may be defined between any of the wells 408 or reaction sites 414 that are positioned directly over a corresponding sensor 440 or pixel.

In some other examples, a single image sensor 440 may receive photons through more than one light guide 462 and/or from more than one reaction site 414. In such versions, the particular region of the single image sensor 440 that is directly aligned with (e.g., positioned directly under) a reaction site 414 may be said to form a “sensing pair” with that reaction site 414. As used herein, a single image sensor 440 may include one pixel or more than one pixel. By way of example only, image sensors 440 may include CCD sensors, CMOS sensors, and/or other kinds of components.

The term “array” or “sub-array” does not necessarily include each and every item of a certain type that the detection device may have. For example, the sensor array 424 may not include each and every image sensor in the detection device 404. Instead, the detection device 404 may include other image sensors (e.g., other array(s) of image sensors). As another example, the guide array 426 may not include each and every light guide of the detection device. Instead, there may be other light guides that are configured differently than the light guides 462 or that have different relationships with other elements of the detection device 404. As such, unless explicitly recited otherwise, the term “array” may or may not include all such items of the detection device.

In the illustrated example, the flow cell 402 includes a sidewall 406 and a flow cover 410 that is supported by the sidewall 406 and other sidewalls (not shown). The sidewalls are coupled to the detector surface 412 and extend between the flow cover 410 and the detector surface 412. In some examples, the sidewalls are formed from a curable adhesive layer that bonds the flow cover 410 to the detection device 404.

The flow cell 402 is sized and shaped so that a flow channel 418 exists between the flow cover 410 and the detection device 404. As shown, the flow channel 418 may include a height H₁. By way of example only, the height H₁ may be between about 50-400 μm (microns) or, more particularly, about 80-200 μm. In the illustrated example, the height H₁ is about 100 μm. The flow cover 410 may include a material that is transparent to excitation light 401 propagating from an exterior of the biosensor 400 into the flow channel 418. As shown in FIG. 3, the excitation light 401 approaches the flow cover 410 at a non-orthogonal angle. However, this is only for illustrative purposes as the excitation light 401 may approach the flow cover 410 from different angles. Excitation light 401 may be generated by one or more light sources within illumination system 109.

Also shown, the flow cover 410 may include inlet and outlet ports 420, 422 that are to fluidically engage other ports (not shown). For example, the other ports may be from a cartridge or a workstation. The flow channel 418 is sized and shaped to direct a fluid along the detector surface 412. The height H₁ and other dimensions of the flow channel 418 may be to maintain a substantially even flow of a fluid along the detector surface 412. The dimensions of the flow channel 418 may also be to control bubble formation.

The sidewalls 406 and the flow cover 410 may be separate components that are coupled to each other. In other examples, the sidewalls 406 and the flow cover 410 may be integrally formed such that the sidewalls 406 and the flow cover 410 are formed from a continuous piece of material. By way of example, the flow cover 410 (or the flow cell 402) may comprise a transparent material, such as glass or plastic. The flow cover 410 may constitute a substantially rectangular block having a planar exterior surface and a planar inner surface that defines the flow channel 418. The block may be mounted onto the sidewalls 406. Alternatively, the flow cell 402 may be etched to define the flow cover 410 and the sidewalls 406. For example, a recess may be etched into the transparent material. When the etched material is mounted to the detection device 404, the recess may become the flow channel 418.

The detection device 404 has a detector surface 412 that may be functionalized (e.g., chemically or physically modified in a suitable manner for conducting designated reactions). For example, the detector surface 412 may be functionalized and may include a plurality of reaction sites 414 having one or more biomolecules immobilized thereto. The detector surface 412 has an array of reaction recesses or open-sided wells 408 defining reaction chambers, such that each of the wells 408 may include one or more of the reaction sites 414. The wells 408 may be defined by, for example, an indent or change in depth along the detector surface 412. In other examples, the detector surface 412 may be substantially planar.

As shown in FIG. 3, the reaction sites 414 may be distributed in a pattern along the detector surface 412. For instance, the reactions sites 414 may be located in rows and columns along the detector surface 412 in a manner that is similar to a microarray. However, it is understood that various patterns of reaction sites may be used. The reaction sites 414 may include biological or chemical substances that emit light signals. For example, the biological or chemical substances of the reaction sites 414 may generate light emissions in response to the excitation light 401. In particular examples, the reaction sites 414 include clusters or colonies of biomolecules (e.g., oligonucleotides) that are immobilized on the detector surface 412, and fluorophores at the reaction sites 414 may emit light in response to the excitation light 401, with such emitted light being indicative of the composition of biomolecules at the reaction sites 414.

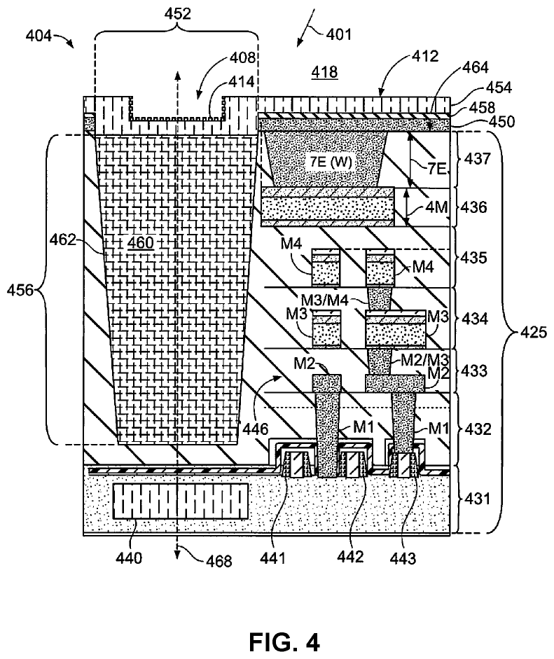

FIG. 4 is an enlarged cross-section of the detection device 404 showing various features in greater detail. More specifically, FIG. 4 shows a single image sensor 440, a single light guide 462 for directing light emissions toward the image sensor 440, and associated circuitry 446 for transmitting signals based on the light emissions (e.g., photons) detected by the image sensor 440. It is understood that the other image sensors 440 of the sensor array 424 (FIG. 3) and associated components may be configured in an identical or similar manner. It is also understood, however, the detection device 404 is not required to be manufactured identically or uniformly throughout. Instead, one or more image sensors 440 and/or associated components may be manufactured differently or have different relationships with respect to one another.

The circuitry 446 may include interconnected conductive elements (e.g., conductors, traces, vias, interconnects, etc.) that are capable of conducting electrical current, such as the transmission of data signals that are based on detected photons. The detection device 404 and/or the device base 425 may comprise an integrated circuit having a planar array of the image sensors 440. The circuitry 446 formed within the detection device 404 may be configured for at least one of signal amplification, digitization, storage, and processing. The circuitry may collect and analyze the detected light emissions and generate data signals for communicating detection data to a bioassay system. The circuitry 446 may also perform additional analog and/or digital signal processing in the detection device 404.

The device base 425 may be manufactured using integrated circuit manufacturing processes, such as processes used to manufacture CMOSs. For example, the device base 425 may include a plurality of stacked layers 431-437 including a sensor layer or base 431, which is a silicon layer or wafer in the illustrated embodiment. The sensor layer 431 may include the image sensor 440 and gates 441-443 that are formed with the sensor layer 431. The gates 441-443 are electrically coupled to the image sensor 440. When the detection device 404 is fully formed as shown in FIGS. 3-4, the image sensor 440 may be electrically coupled to the circuitry 446 through the gates 441-443.

As used herein, the term “layer” is not limited to a single continuous body of material unless otherwise noted. For example, the sensor layer 431 may include multiple sub-layers that are different materials and/or may include coatings, adhesives, and the like. Furthermore, one or more of the layers (or sub-layers) may be modified (e.g., etched, deposited with material, etc.) to provide the features described herein.

In some versions, each image sensor 440 has a detection area that is less than about 50 μm². In particular versions, the detection area is less than about 10 μm². In more particular versions, the detection area is about 2 μm². In such cases, the image sensor 440 may constitute a single pixel. An average read noise of each pixel in an image sensor 440 may be, for example, less than about 150 electrons. In more particular versions, the read noise may be less than about 5 electrons. The resolution of the array of image sensors 440 may be greater than about 0.5 megapixels (MP). In more specific versions, the resolution may be greater than about 5 MP; and, more particularly, greater than about 10 MP.

The device layers also include a plurality of metal-dielectric layers 432-437, which are hereinafter referred to as substrate layers. In the illustrated examples, each of the substrate layers 432-437 includes metallic elements (e.g., W (tungsten), Cu (copper), or Al (aluminum)) and dielectric material (e.g., SiO₂). Various metallic elements and dielectric material may be used, such as those suitable for integrated circuit manufacturing. However, in other versions, one or more of the substrate layers 432-437 may include only dielectric material, such as one or more layers of SiO₂.

With respect to the specific versions shown in FIG. 4, the first substrate layer 432 may include metallic elements referred to as M1 that are embedded within dielectric material (e.g., SiO₂). The metallic elements M1 comprise, for example, W (tungsten). The metallic elements M1 extend entirely through the substrate layer 432 in the illustrated version. The second substrate layer 433 includes metallic elements M2 and dielectric material as well as a metallic interconnect (M2/M3). The third substrate layer 434 includes metallic elements M3 and metal interconnects (M3/M4). The fourth substrate layer 435 also includes metallic elements M4. The device base 425 also includes fifth and sixth substrate layers 436, 437.

As shown, the metallic elements and interconnects are connected to each other to form at least a portion of the circuitry 446. In the illustrated version, the metallic elements M1, M2, M3, M4 include W (tungsten), Cu (copper), and/or aluminum (Al) and the metal interconnects M2/M3 and M3/M4 include W (tungsten), but it is understood that other materials and configurations may be used. It is also noted that the device base 425 and the detection device 404 shown in FIGS. 3-4 are for illustrative purposes only. For example, other versions may include fewer or additional layers than those shown in FIGS. 3-4 and/or different configurations of metallic elements.

In some versions, the detection device 404 includes a shield layer 450 that extends along an outer surface 464 of the device base 425. In the illustrated version, the shield layer 450 is deposited directly along the outer surface 464 of the substrate layer 437. However, an intervening layer may be disposed between the substrate layer 437 and the shield layer 450 in other versions. The shield layer 450 may include a material that is configured to block, reflect, and/or significantly attenuate the light signals that are propagating from the flow channel 418. The light signals may be the excitation light 401 and/or the light emissions generated by biological or chemical substances at the reaction sites 414 in response to the excitation light 401. By way of example only, the shield layer 450 may comprise tungsten (W).

As shown in FIG. 4, the shield layer 450 of the present example includes an aperture or opening 452 therethrough. The shield layer 450 may include an array of such apertures 452. In some versions, the shield layer 450 may extend continuously between adjacent apertures 452. In such versions, the light signals from the flow channel 418 may be blocked, reflected, and/or significantly attenuated to prevent detection of such light signals by the image sensors 440. However, in other versions, the shield layer 450 does not extend continuously between the adjacent apertures 452 such then one or more openings other than the apertures 452 exits in the shield layer 450.

The detection device 404 may also include a passivation layer 454 that extends along the shield layer 450 and across the apertures 452. The shield layer 450 may extend over the apertures 452 thereby directly or indirectly covering the apertures 452. The shield layer 450 may be located between the passivation layer 454 and the device base 425. An adhesive or promoter layer 458 may be located therebetween to facilitate coupling the passivation layer 454 and shield layer 450. The passivation layer 454 may be configured to protect the device base 425 and the shield layer 450 from the fluidic environment of the flow channel 418.

In some cases, the passivation layer 454 may also be configured to provide a solid surface (e.g., the detector surface 412) that permits biomolecules or other analytes-of-interest to be immobilized thereon. For example, each of the reaction sites 414 may include a cluster of biomolecules that are immobilized to the detector surface 412 of the passivation layer 454. Thus, the passivation layer 454 may be formed from a material that permits the reaction sites 414 to be immobilized thereto. The passivation layer 454 may also comprise a material that is at least transparent to a desired fluorescent light. By way of example, the passivation layer 454 may include silicon nitride (Si₃N₄) and/or silica (SiO₂). However, other suitable material(s) may be used. In addition, the passivation layer 454 may be physically or chemically modified to facilitate immobilizing the biomolecules and/or to facilitate detection of the light emissions.

In the illustrated version, a portion of the passivation layer 454 extends along the shield layer 450 and a portion of the passivation layer 454 extends directly along filter material 460 of a light guide 462. The reaction recess 408 may be formed directly over the light guide 462. In some cases, prior to the passivation layer 454 being deposited along the shield layer 450 or adhesion layer 458, a base hole or cavity 456 may be formed within the device base 425. For example, the device base 425 may be etched to form an array of the base holes 456. In particular versions, the base hole 456 is an elongated space that extends from proximate the aperture 452 toward the image sensor 440. The base hole may extend lengthwise along a central longitudinal axis 468. A three-dimensional shape of the base hole 456 may be substantially cylindrical or frusto-conical in some embodiments, such that a cross-section taken along a plane that extends into the page of FIG. 4 is substantially circular. The longitudinal axis 468 may extend through a geometric center of the cross-section. However, other geometries may be used in alternative versions. For example, the cross-section may be substantially square-shaped or octagonal.

The filter material 460 may be deposited within the base hole 456 after the base hole 456 is formed. The filter material 460 may form (e.g., after curing) a light guide 462. The light guide 462 is configured to filter the excitation light 401 and permit the light emissions 466 to propagate therethrough toward the corresponding image sensor 440. The light guide 462 may include, for example, an organic absorption filter. By way of specific example only, the excitation light may be about 532 nm and the light emissions may be about 570 nm or more.

In some cases, the organic filter material of the light guide 462 may be incompatible with other materials of the biosensor 400. For example, the organic filter material may have a coefficient of thermal expansion that causes the filter material to significantly expand. Alternatively, or additionally, the filter material may be unable to sufficiently adhere to certain layers, such as the shield layer 450 (or other metal layers). Expansion of the filter material may cause mechanical stress on the layers that are adjacent to the filter material or structurally connected to the filter material. In some cases, the expansion may cause cracks or other unwanted features in the structure of the biosensor. Thus, versions set forth herein may limit the degree to which the filter material expands and/or the degree to which the filter material is in contact with other layers. For example, the filter material of different light guides 462 may be isolated from each other by the passivation layer 454. In such versions, the filter material may not contact the metal layer(s). Moreover, the passivation layer 454 may resist expansion and/or permit some expansion while reducing generation of unwanted structural features (e.g., cracks).

The light guide 462 may be configured relative to surrounding material of the device base 425 (e.g., the dielectric material) to form a light-guiding structure. For example, the light guide 462 may have a refractive index of about 2.0 so that the light emissions are substantially reflected at an interface between the light guide 462 and the material of the device base 425. In certain versions, the light guide 462 is configured such that the optical density (OD) or absorbance of the excitation light is at least about 4 OD. More specifically, the filter material may be selected and the light guide 462 may be dimensioned to achieve at least 4 OD. In more particular versions, the light guide 462 may be configured to achieve at least about 5 OD or at least about 6 OD.

III. Example of Flow Cell with Full Curtains

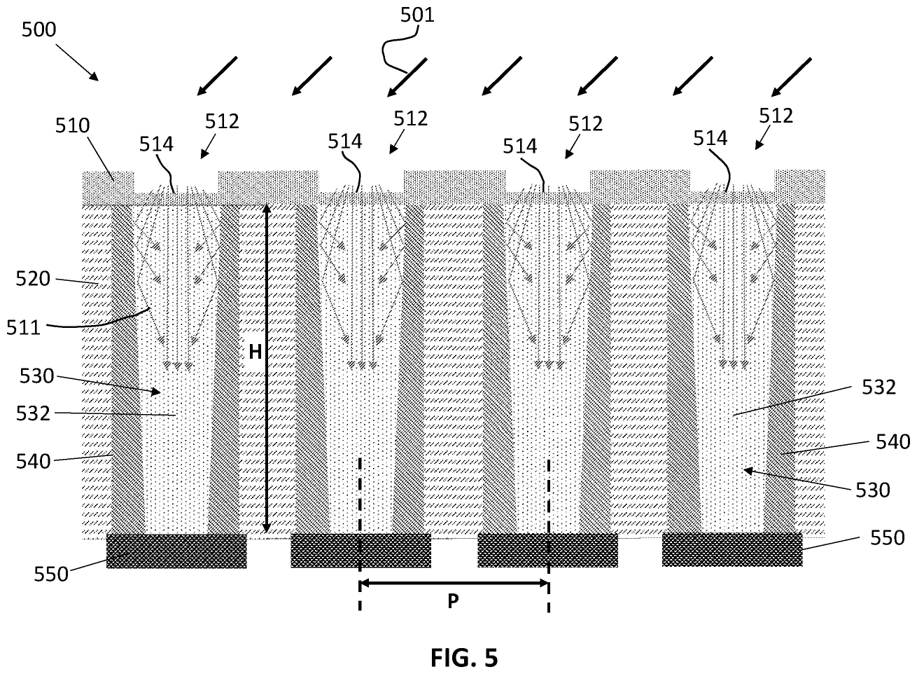

In some versions of biosensor 400, each light guide 462 may be lined with an opaque material, such as one or more metals. An example of such an arrangement is shown in FIG. 5. In particular, FIG. 5 shows a biosensor 500 that includes a flow channel floor 510 defining a plurality of wells 512, with each well 512 providing a reaction site 514. A base 520 underneath the floor 510 defines a plurality of light guides 530, with each light guide 530 being positioned under a corresponding reaction site 514. Each light guide 530 contains a filter material 532. Each light guide 530 also has a tapered profile in this example, such that the upper region of light guide 530 is wider than the lower region of light guide 530, with the width linearly narrowing from the upper region to the lower region.

As biosensor 500 is exposed to excitation light 501 (e.g., as generated by one or more light sources within illumination system 109), the excitation light 501 causes fluorophores at reaction sites 514 to emit light 511. The filter material 532 filters out the excitation light 501 without filtering out the emitted light 511. In scenarios where nucleic acids are at reaction sites 514, the emitted light 511 may indicate the composition of such nucleic acids. An image sensor 550 is positioned under each light guide 530 and is configured to receive the light 511 emitted from the corresponding reaction site 514 via the corresponding light guide 530. Thus, each image sensor 550 forms a “sensing pair” with the reaction site 514 that is directly aligned with (e.g., positioned directly above) the image sensor 550. In versions where each image sensor 550 represents a single pixel, the image sensor 550 forming a sensing pair with a reaction site 514 may be referred to as the “center pixel” associated with that reaction site 514; while the image sensors 550 adjacent to the center pixel may be referred to as “neighbor pixels.” Similarly, an image sensor 550 that does not form a sensing pair with a given reaction site 514 may be referred to as a “neighbor sensor” with respect to that reaction site 514.

In some other examples, a single image sensor 550 may receive photons through more than one light guide 530 and/or from more than one reaction site 514. In such versions, the particular region of the single image sensor 550 that is directly aligned with (e.g., positioned directly under) a reaction site 514 may be said to form a “sensing pair” with that reaction site 514.

As shown in FIG. 5, biosensor 500 provides a height distance (H) between each image sensor 550 and the underside of the floor 510 in the region underneath the reaction site 514 forming a sensing pair with that image sensor 550. In this example, this height distance (H) represents the thickness of base 520. By way of example only, this height distance (H) may range from approximately 2 micrometers to approximately 4 micrometers; or may be approximately 3.5 micrometers.

Alternatively, biosensor 500 may provide any other suitable height distance (H). As also shown in FIG. 5, biosensor 500 provides a pitch distance (P) that is defined between a central axis of an image sensor 550 and each adjacent image sensor 500. This pitch distance (P) also represents the distance between a central axis of a well 512 and each adjacent well 512. By way of example only, this pitch distance (P) may range from approximately 0.7 micrometers to approximately 2.0 micrometers; or may be approximately 1 micrometer. Alternatively, biosensor 500 may provide any other suitable pitch distance (P).

The components of biosensor 500 that are described above may be configured and operable like the similar components described above with respect to biosensor 400. Moreover, biosensor 500 may include additional components such as any of those additional components described above in the context of biosensor 400 even if such additional components are not depicted in FIG. 5.

Unlike the biosensor 400 depicted in FIGS. 3-4, the biosensor 500 depicted in FIG. 5 includes a plurality of shields or curtains 540. Each curtain 540 surrounds a corresponding light guide 530 and extends the full vertical height of base 520, such that each curtain 540 extends from a corresponding image sensor 550 to floor 510. Curtains 540 thus define interruptions along the width of base 520. Curtains 540 also fully contain corresponding volumes of filter material 532, such that no portions of filter material 532 span across the full width of base 520. Curtains 540 of this example are formed of an opaque material such as metal, though curtains 540 may alternatively be formed of other materials or combinations of materials. Curtains 540 are configured to prevent light 511 emitted at one reaction site 514 from reaching an image sensor 550 that is positioned directly under another reaction site 514. In other words, curtains 540 prevent light 511 emitted at a reaction site 514 from reaching image sensors 550 that do not form a sensing pair with that reaction site 514. Curtains 540 thus ensure that light 511 emitted at a given reaction site 514 is only received by the image sensor 550 forming a sensing pair with that reaction site 514.

In doing so, curtains 540 prevent the occurrence of optical crosstalk within biosensor 500.

As used herein, the term “crosstalk” may be read to include the proportion of optical signals from a given reaction site 514 reaching image sensors 550 that do not form a sensing pair with the reaction site. In versions where each image sensor 550 represents a single pixel, crosstalk may be understood to mean the proportion of optical signals reaching all pixels other than the center pixel.

IV. Example of Loss-Induced Crosstalk Reduction in Biosensor

As described above, the integration of curtains 540 into a biosensor 500 may effectively prevent optical crosstalk within the biosensor 500 by preventing light 511 emitted at a reaction site 514 from reaching an image sensor 550 that does not form a sensing pair with the reaction site 514. However, including curtains 540 in a biosensor 500 may tend to add complexity and expense to the process of manufacturing biosensor 500, especially with curtains 540 extending through the full height distance (H) of biosensor 500. Such complexity and expense may be due, at least in part, to curtains 540 having sub-micron feature sizes (in the x-y plane) and several-micron thickness (in the z direction). Such complexity and expense may also be due, at least in part, to filter material 460 being applied separately within each individual light guide 462.

In addition, it may be desirable to minimize the pitch distance (P) in a biosensor 500 in order to maximize the total number of reaction sites 514 in the biosensor 500 (i.e., to maximize the density of reaction sites 514 in biosensor 500); and the presence of curtains 540 in a biosensor 500 may constrain the reduction of pitch distance (P) in the biosensor 500 since curtains 540 occupy physical space in the biosensor. In other words, it may be possible to reduce the pitch distance (P) in the biosensor 500 if curtains 540 were to be eliminated.

It may therefore be desirable to provide a version of a biosensor that suitably prevents or reduces the occurrence of optical crosstalk, without presenting the manufacturing complexity and expense associated with curtains 540; and without constraining the reduction of pitch distance (P) in the biosensor in the way that curtains 540 constrain the reduction of pitch distance (P). The following examples provide versions of a biosensor that may suitably prevent or reduce the occurrence of optical crosstalk, without presenting the manufacturing complexity and expense associated with curtains 540; and without constraining the reduction of pitch distance (P) in the biosensor as may otherwise occur when curtains 540 are present. In particular, instead of physically constraining transmission of light by physically blocking the light as is done by curtains 540, examples described below provide tailored absorption of light that might otherwise result in crosstalk. Such tailored absorption of light may be referred to as loss-induced crosstalk reduction or “LICR.” To the extent that the LICR features described below do not completely eliminate crosstalk, the LICR features described below may at least reduce the crosstalk to a degree where any remaining crosstalk may be computationally corrected through conventional image processing techniques (where such image processing techniques, alone, may be insufficient in the absence of the LICR features described below).

A. Example of Biosensor without Curtains and with Crosstalk

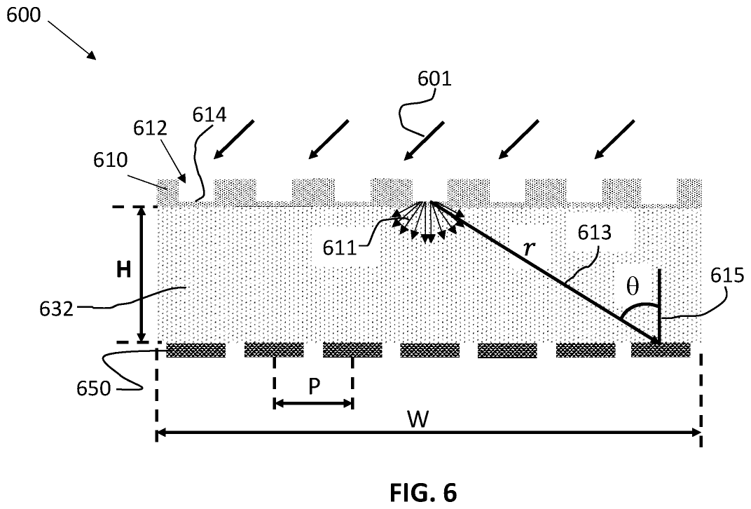

FIG. 6 shows an example of a biosensor 600 that lacks curtains like curtains 540 of biosensor 500. Biosensor 600 of this example includes a flow channel floor 610 defining a plurality of wells 612, with each well 612 providing a reaction site 614. A layer 632 of filter material is positioned under flow channel floor 610. A plurality of image sensors 650 are positioned under the layer 632 of filter material. Each image sensor 650 is vertically centered under a corresponding well 612 and reaction site 614, such that each sensor 650 forms a sensing pair with a corresponding reaction site 614. In this example, the layer 632 of filter material in biosensor 600 effectively forms a structural equivalent of base 520 in biosensor 500. The layer 632 of filter material spans the full height distance (H) and width distance (W) of biosensor 600.

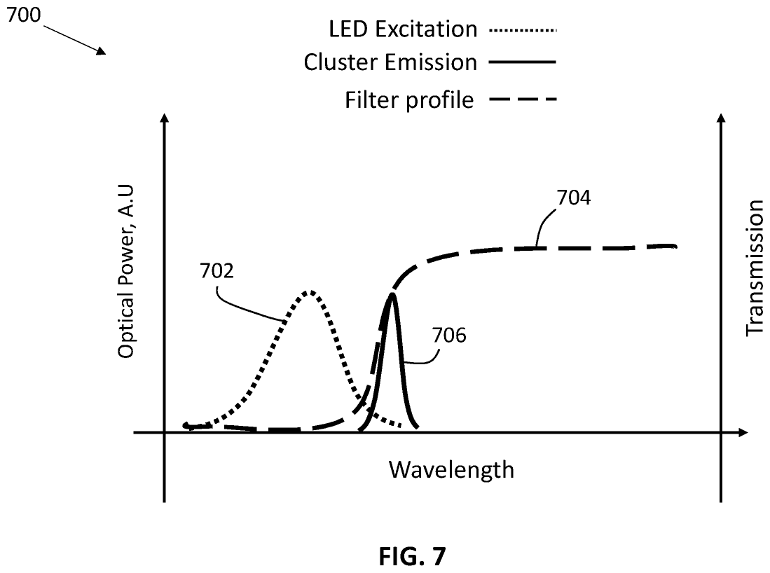

As biosensor 600 is exposed to excitation light 601 (e.g., as generated by one or more light sources within illumination system 109), the excitation light 601 causes fluorophores at reaction sites 614 to emit light 611. In scenarios where nucleic acids are at reaction sites 614, the emitted light 611 may indicate the composition of such nucleic acids. Image sensors 650 receive the light 611 emitted from the reaction sites 614 via the layer 632 of filter material. The filter material of layer 632 filters out the excitation light 601 without filtering out the emitted light 611. An example of this filtering is shown in the graph 700 depicted in FIG. 7. In particular, FIG. 7 depicts a plot 702 of the excitation light 601 in terms of power over wavelength, a plot 704 of the filter profile of the layer 632 of filter material in terms of transmission over wavelength, and a plot 706 of the emitted light 611 in terms of power over wavelength. As shown, the layer 632 of filter material prevents transmission of substantially all wavelengths of excitation light 601 while permitting transmission of all wavelengths of emitted light 611.

Since biosensor 600 of the example shown in FIG. 6 lacks light-blocking features like curtains 540, and since the filter material of layer 632 is not configured to filter emitted light 611, emitted light 611 from any given reaction site 614 may reach one or more image sensors 650 that do not form a sensing pair with the reaction site 614. In other words, emitted light 611 from any given reaction site 614 may reach one or more image sensors 650 that are not directly underneath the reaction site 614. Thus, biosensor 600 generates crosstalk as emitted light 611 from a given reaction site 614 propagates through layer 632 of filter material at non-vertical angles to reach various image sensors 650 that do not form a sensing pair with the reaction site 614. In other words, biosensor 600 generates crosstalk as emitted light 611 from a given reaction site 614 propagates through layer 632 of filter material at non-vertical angles to reach image sensors 650 that are not directly below the reaction site 614. FIG. 6 shows such crosstalk occurring along an optical path having a length (r) and defining an angle (Θ) with an axis 615 that is normal to the image sensor 650 receiving the light 611.

The distribution of an optical signal from light 611 emitted from a single reaction site 614 over the image sensors 650 of biosensor 600 may be defined as a point-spread function (PSF). The PSF may thus represent the degree of crosstalk occurring within biosensor 600. The PSF may depend on the height-to-pitch ration (H/P), as shown below in equation (I):

$$PSF ( r , θ ) α \frac{cos ( θ )}{{r}^{2}} = \frac{H}{{r}^{3}} \qquad ( I )$$

where “PSF” is the point spread function;
“r” is the length of the optical path between the reaction site 614 from which the light 611 is being emitted;
“Θ” is the angle defined between the optical path of “r” and an axis 615 that is normal to the image sensor 650 receiving the emitted light 611; and
“H” is the height of the layer 632 of filter material.

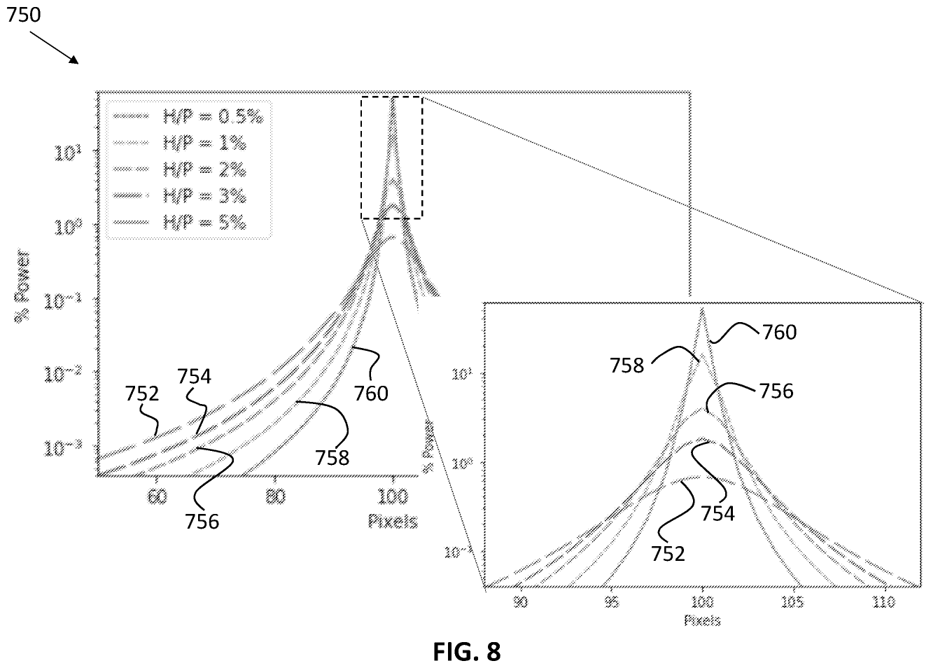

It should be understood that the value of “r” and “Θ” may vary based on the pitch distance (P) as defined above, such that the PSF will ultimately depend on the height-to-pitch ratio (H/P) of biosensor 600. FIG. 8 depicts a graph 750 showing plots 752, 754, 756, 758, 760 of different examples of PSFs based on different H/P values. For instance, plot 752 shows a PSF for a version of biosensor 600 having a H/P value of 5. Plot 754 shows a PSF for a version of biosensor 600 having a H/P value of 3. Plot 756 shows a PSF for a version of biosensor 600 having a H/P value of 2. Plot 758 shows a PSF for a version of biosensor 600 having a H/P value of 1. Plot 760 shows a PSF for a version of biosensor 600 having a H/P value of 0.5.

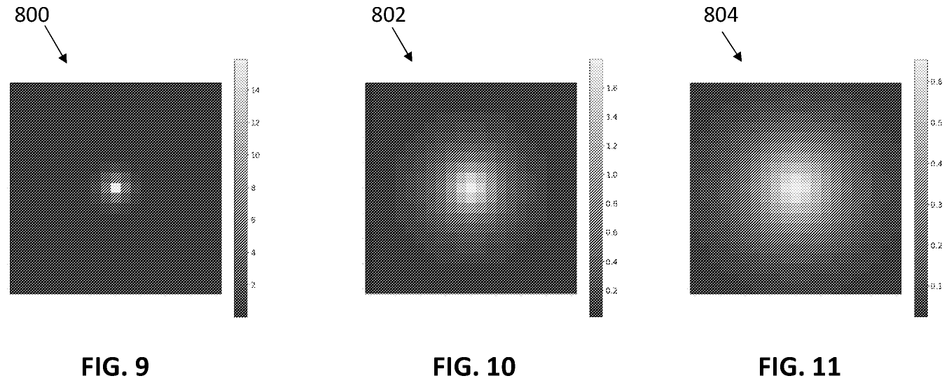

FIGS. 9-11 also show examples of images 800, 802, 804 captured by image sensors 650 of biosensor 600, representing PSFs associated with light 611 emitted from a central reaction site 614 in variations of biosensor 600 having different H/P ratio values. In particular, FIG. 9 shows an example of an image 800 captured by image sensors 650 of a version biosensor 600 having a H/P of 1, where the image shows the PSF associated with light 611 emitted from a given reaction site 614. FIG. 10 shows an example of an image 802 captured by image sensors 650 of a version biosensor 600 having a H/P of 3, where the image shows the PSF associated with light 611 emitted from a given reaction site 614. FIG. 10 shows an example of an image 800 captured by image sensors 650 of a version biosensor 600 having a H/P of 5, where the image shows the PSF associated with light 611 emitted from a given reaction site 614.

B. Example of Biosensor without Curtains and with LICR

As can be seen from the examples provided in FIGS. 8-11, the larger the H/P ratio, the larger or wider the PSF. A larger or wider the PSF may be viewed as representing a larger degree of more crosstalk. Thus, it may be desirable to minimize the PSF. As noted above, it may also be desirable to minimize the pitch distance (P) in order to maximize the number or density of reaction sites in a biosensor. In view of this, in order to minimize the H/P ratio to thereby minimize the PSF, while also minimizing the pitch distance (P), it may seem like a clear solution would be to also minimize the height distance (H). However, other considerations may prevent such changes to the configuration of a biosensor. By way of example only, the structural configuration of system receptacle 112 and/or other components of system 100 may require a biosensor to have a certain thickness or at least a minimum thickness; and such requirements may constrain the ability to reduce the height distance (H) of the biosensor. Still other practical considerations may prevent reductions in the height distance (H) of the biosensor. It may therefore be desirable to find another way to reduce crosstalk (i.e., to minimize the PSF) without changing the height distance (H); and without introducing the drawbacks described above with respect to curtains 540 that extend along the full height distance (H).

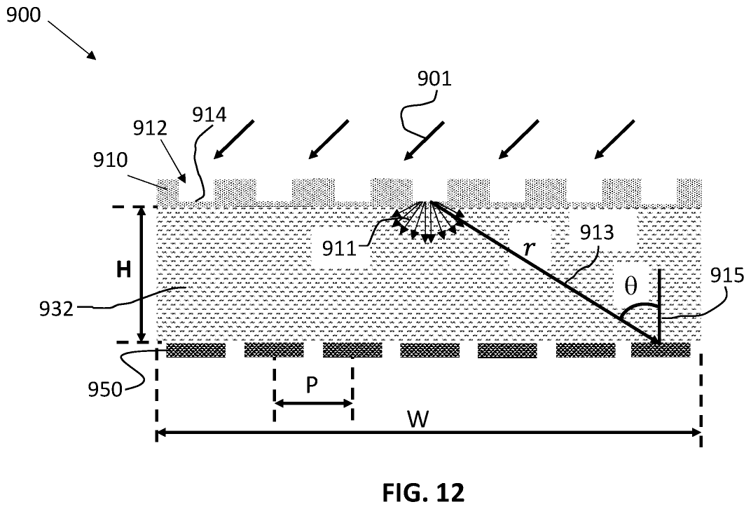

FIG. 12 shows an example of a biosensor 900 that is configured to provide LICR to thereby reduce crosstalk (i.e., to minimize the PSF), without changing the height distance (H); and without introducing the drawbacks described above with respect to curtains 540 that extend along the full height distance (H). Biosensor 900 may be used in bioassay system 100 as a version of biosensor 102. Biosensor 900 of this example includes a flow channel floor 910 defining a plurality of wells 912, with each well 912 providing a reaction site 914. A layer 932 of filter material is positioned under flow channel floor 910. A plurality of image sensors 950 are positioned under the layer 932 of filter material. In some versions, image sensors 950 and layer 932 are formed together as a single monolithic component. Each image sensor 950 is vertically centered under a corresponding well 912 and reaction site 914, such that each sensor 950 forms a sensing pair with a corresponding reaction site 914. In this example, the layer 932 of filter material in biosensor 900 effectively forms a structural equivalent of base 520 in biosensor 500. The layer 932 of filter material spans the full height distance (H) and width distance (W) of biosensor 900.

As biosensor 900 is exposed to excitation light 901 (e.g., as generated by one or more light sources within illumination system 109), the excitation light 901 causes fluorophores at reaction sites 914 to emit light 911. In scenarios where nucleic acids are at reaction sites 914, the emitted light 911 may indicate the composition of such nucleic acids. Image sensors 950 receive the light 911 emitted from the reaction sites 914 via the layer 932 of filter material.

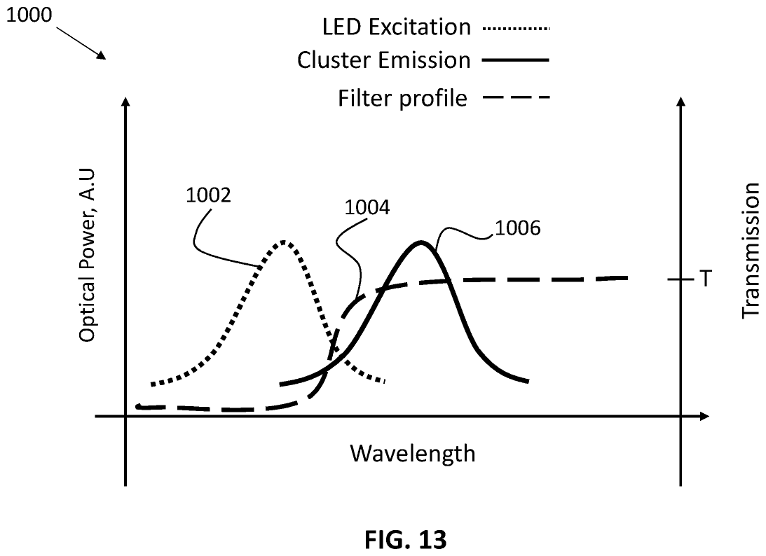

Unlike the filter material of layer 632 described above, the filter material of layer 932 in biosensor 900 filters out some of the emitted light 911 in addition to filtering out the excitation light 901. In the sense that the purpose of image sensors 950 is to detect emitted light 911, the intentional filtering of emitted light 911 may be considered counterintuitive, as this may seem to reduce the sensitivity of the biosensor 900. An example of this intentional filtering of emitted light 911 is shown in the graph 1000 depicted in FIG. 13. In particular, FIG. 13 depicts a plot 1002 of the excitation light 901 in terms of power over wavelength, a plot 1004 of the filter profile of the layer 932 of filter material in terms of transmission over wavelength, and a plot 1006 of the emitted light 911 in terms of power over wavelength. As shown, the layer 932 of filter material prevents transmission of substantially all wavelengths of excitation light 901, prevents transmission of some wavelengths of emitted light 911, and permits transmission of some other wavelengths of emitted light 911.

In filtering out some wavelengths of emitted light 911, the layer 932 of filter material may reduce the ability of light 911 emitted from a given reaction site 914 to reach image sensors 950 that do not form a sensing pair with that reaction site 914. The transmission (T) may be exponentially reduced by absorption over the optical path length (r) with a material-dependent characteristic length (a). Because the optical path length (r) to a neighbor sensor 950 is always greater than the path length (r) to the center sensor 950, the potential signal at any neighbor sensor 950 (i.e., the crosstalk) is always reduced by absorption of emitted light 911 in the layer 932. The fluorescence PSF is thus reduced in width or “squeezed” by absorption of emitted light 911 in the layer 932. This effect is shown below in equation (II):

$$PSF ( r , θ ) α \frac{cos ( θ )}{{r}^{2}} Loss ( r ) = \frac{H}{{r}^{3}} {T}^{- r / H} \qquad ( II )$$

where “PSF” is the point spread function;
“r” is the length of the optical path between the reaction site 914 from which the light 911 is being emitted;
“Θ” is the angle defined between the optical path of “r” and an axis 915 that is normal to the image sensor 950 receiving the emitted light 911;
“H” is the height of the layer 932 of filter material; and
“T” is the transmission over the height distance (H).

The value for “T” may be calculated using the following formula (III):

T=e^{-αH} (III)

where “T” is the transmission over the height distance (H);
“e” is Euler's number;
“α” is the absorption coefficient in emitted light 911 wavelength; and
“H” is the height of the layer 932 of filter material.

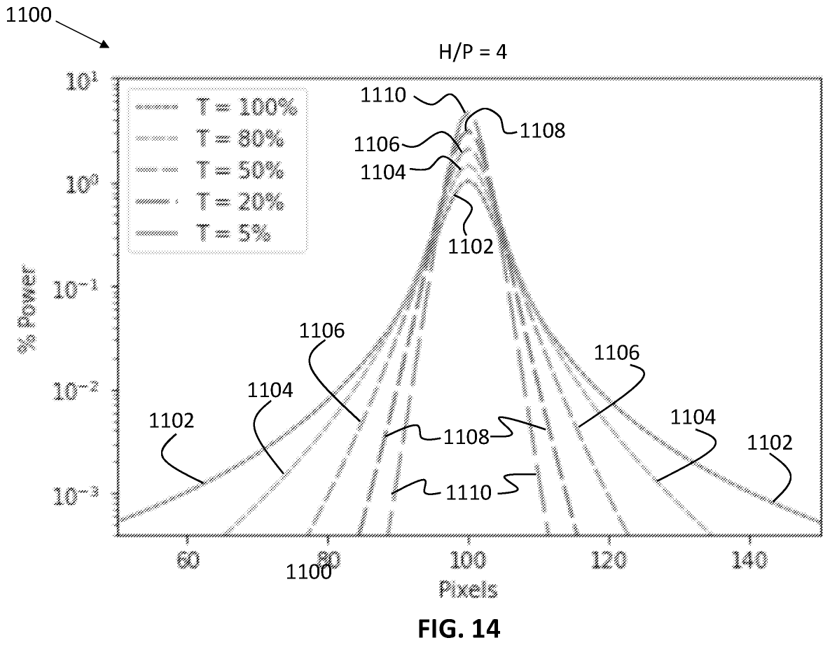

It should be understood that reducing the transmission value (T) may provide a corresponding reduction in the PSF width. An example of this is shown in FIG. 14, which depicts a graph 1100 showing plots 1102, 1104, 1106, 1108, 1110 of different examples of PSFs based on different transmission values (T). In each of these examples depicted in FIG. 14, the H/P value is 4. Plot 1102 shows a PSF for a version of biosensor 900 having transmission value (T) of 1.00 (or 100%). Plot 1104 shows a PSF for a version of biosensor 900 having transmission value (T) of 0.80 (or 80%). Plot 1106 shows a PSF for a version of biosensor 900 having transmission value (T) of 0.50 (or 50%). Plot 1108 shows a PSF for a version of biosensor 900 having transmission value (T) of 0.20 (or 20%). Plot 1110 shows a PSF for a version of biosensor 900 having transmission value (T) of 0.05 (or 5%).

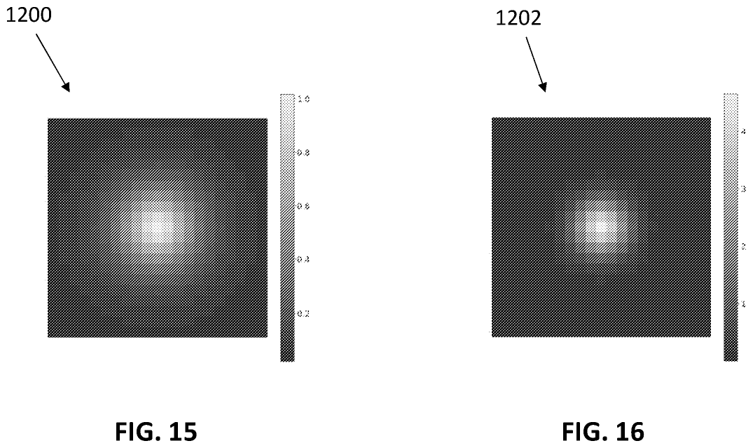

As can be seen through the plots 1102, 1104, 1106, 1108, 1110 in FIG. 14, reducing the transmission value (T) willer duce the PSF width. As noted above, reducing the PSF width may represent a corresponding reduction in crosstalk. An example of this is depicted in FIGS. 15-16, where FIG. 15 shows an example of an image 1200 captured by an image sensor 950 of a version of biosensor 900 having a transmission value (T) of 1.00 (or 100%); while FIG. 16 shows an example of an image 1202 captured by an image sensor 950 of a version of biosensor 900 having a transmission value (T) of 0.05 (or 5%). Each image 1200, 1202 represents emitted light 911 captured by all image sensors 950 of biosensor 900, where the light 911 is emitted only by one reaction site 914 at the center of the biosensor 900. The image 1200 of FIG. 15 may represent a signal-to-background ratio of approximately 1/99 or 1.0%. The image 1202 of FIG. 15 may represent a signal-to-background ratio of approximately 6/94 or 6.4%.

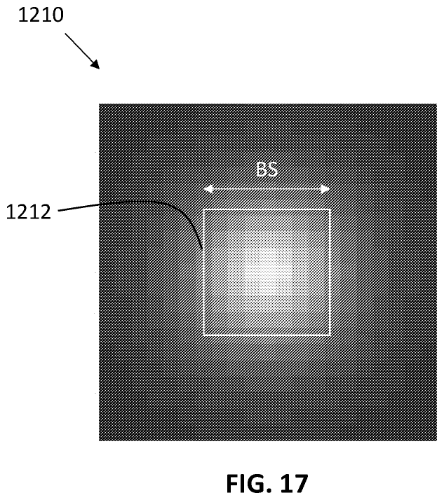

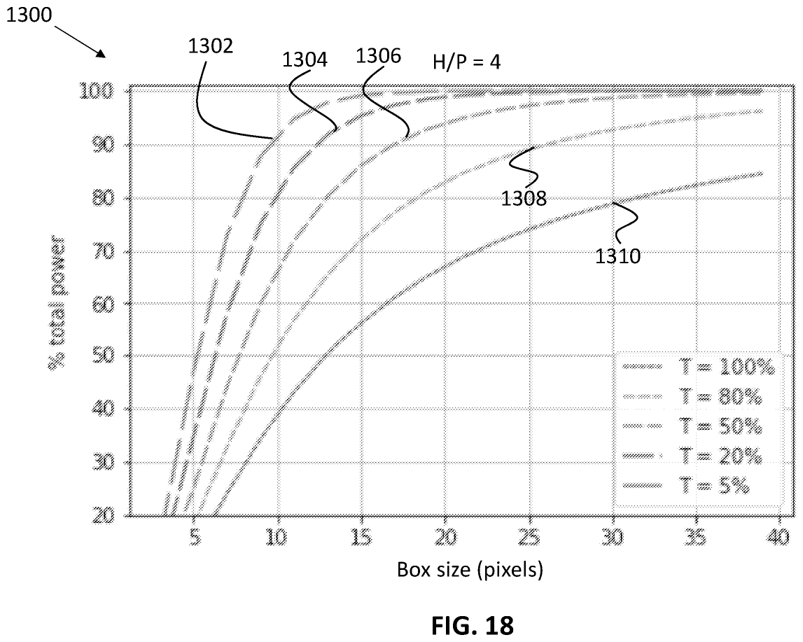

FIGS. 17-18 provide another illustration of how transmission value (T) may affect crosstalk in biosensor 900. FIG. 17 shows an example of an image 1210 representing emitted light 911 captured by all image sensors 950 of biosensor 900, where the light 911 is emitted only by one reaction site 914 at the center of the biosensor 900. A box 1212 in the middle of image 1210 represents a reference region of image 1210 corresponding to the spread of the emitted light 911 as captured by the image sensors 950. The box 1212 has a box size (BS) that may be considered in the context of FIG. 18. In particular, FIG. 18 shows a graph 1300 with several plots 1302, 1304, 1306, 1308, 1310 of different examples of the power of optical signals received by image sensors 950 as a function of the box size (BS) of box 1212. In each of these examples depicted in FIG. 18, the H/P value is 4. Plot 1310 shows the percentage of signal power in relation to the box size (BS) in a version of biosensor 900 having a transmission value (T) of 1.00 (or 100%). Plot 1308 shows the percentage of signal power in relation to the box size (BS) in a version of biosensor 900 having transmission value (T) of 0.80 (or 80%). Plot 1306 shows the percentage of signal power in relation to the box size (BS) in a version of biosensor 900 having transmission value (T) of 0.50 (or 50%). Plot 1304 shows the percentage of signal power in relation to the box size (BS) in a version of biosensor 900 having transmission value (T) of 0.20 (or 20%). Plot 1302 shows the percentage of signal power in relation to the box size (BS) in a version of biosensor 900 having transmission value (T) of 0.05 (or 5%).

Since using a filter material for layer 932 that will filter out some of the emitted light 911 that is intended to be captured by an image sensor 950 that is directly under the reaction site from which the light 911 is emitted, it may be desirable to achieve a certain tradeoff when determining a suitable transmission value (T). This tradeoff may be to provide enough filtering of emitted light 911 to meaningfully reduce crosstalk while still permitting enough of the emitted light 911 to reach the image sensor 950 that is directly under the reaction site from which the light 911 is emitted. In this context, “enough” of the emitted light 911 would be an amount of emitted light 911 that is sufficient to generate a signal at image sensor 950 that allows analysis module 138 to reliably determine the nucleotide sequence of a substance (or other aspects of other compositions) on the reaction site 914 forming a sensing pair with that image sensor 950. In some scenarios, the signal at image sensors 950 may be enhanced by increasing integration time, which may include the time period during which reaction sites 914 are illuminated and emitted light 911 is collected at image sensors 950. In addition, or in the alternative, the signal at image sensors 950 may be enhanced by increasing brightness of the clusters.

Thus, the transmission value (T) should be high enough to permit each image sensor 950 to receive enough emitted light from the reaction site 914 that is directly above the image sensor 950 to generate a meaningful signal; while being low enough to result in a PSF width representing a minimum degree of crosstalk. Such a minimum degree of crosstalk need not necessarily be zero crosstalk; but may be a low enough degree of crosstalk to allow such crosstalk to be readily accounted for through image processing techniques. In some versions, the transmission value (T) ranges from approximately 0.20 (or approximately 20%) to approximately 0.40 (or approximately 40%). In some other versions, the transmission value (T) is as low as 0.10 (or approximately 10%) or even 0.01 (or approximately 1%). Alternatively, any other suitable transmission value (T) may be used. Examples of image processing techniques that may be used to account for any crosstalk that does occur are described in U.S. Provisional Pat. App. No. 63/221,236, entitled “Methods and Systems for Real Time Extraction of Crosstalk in Illumination Emitted from Reaction Sites,” filed Jul. 13, 2021, the disclosure of which is incorporated by reference herein, in its entirety; and U.S. Provisional Pat. App. No. 63/216,125, entitled “Methods and Systems to Correct Crosstalk in Illumination Emitted from Reaction Sites,” filed Jun. 29, 2021, the disclosure of which is incorporated by reference herein, in its entirety.

As described above, the filter material of layer 932 may provide relatively high absorption of wavelengths of excitation light 901 while providing relatively moderate absorption of wavelengths of emitted light 911. In some versions, the transmission of excitation light 901 through layer 932 may be at least approximately 10⁷ less than the transmission of emitted light 911 through layer 932. Alternatively, any other suitable relationship may be provided between transmission of excitation light 901 through layer 932 and transmission of emitted light 911 through layer 932. Regardless of the materials that are used, some methods of manufacture may include spin-coating the material of layer 932 onto a substrate containing image sensors 950. Alternatively, any other suitable methods may be used.

C. Examples of Biosensor with Partial Curtains and with LICR

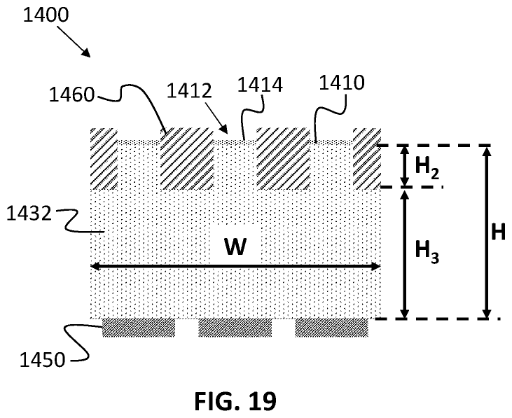

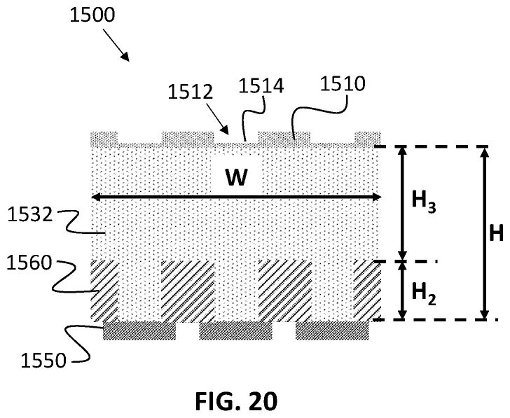

While biosensor 900 lacks any curtains between flow channel floor 910 and image sensors 950, some variations of biosensor 900 may include partial curtains. Examples of such variations are shown in FIGS. 19-20. In particular, FIG. 19 shows a biosensor 1400 that includes a flow channel floor 1410 defining a plurality of wells 1412, with each well 1412 providing a reaction site 1414. Biosensor 1400 may be used in bioassay system 100 as a version of biosensor 102. A layer 1432 of filter material is positioned under flow channel floor 1410. A plurality of image sensors 1450 are positioned under the layer 1432 of filter material. In some versions, image sensors 1450 and layer 1432 are formed together as a single monolithic component. Each image sensor 1450 is vertically centered under a corresponding well 1412 and reaction site 1414, such that each sensor 1450 forms a sensing pair with a corresponding reaction site 1414. In this example, the layer 1432 of filter material in biosensor 1400 effectively forms a structural equivalent of base 520 in biosensor 500. The layer 1432 of filter material spans the full height distance (H) and width distance (W) of biosensor 1400. The layer 1432 of filter material in biosensor 1400 may be configured and operable like the layer 932 of filter material in biosensor 900 as described above, such that the layer 1432 of filter material may provide LICR effects as described above.

Unlike biosensor 900, biosensor 1400 of this example includes a plurality of partial shields or curtains 1460. Except as described below, partial curtains 1460 may be configured and operable like curtains 540 described above. Partial curtains 1460 are positioned between adjacent wells 1412 and extend through a first portion (H₂) of the height distance (H). Thus, a second portion (H₃) of the height distance (H) remains without any partial curtains 1460 extending therethrough. In other words, the layer 1432 of filter material still spans the full width distance (W) of biosensor 1400 within the second portion (H₃) of the height distance (H). In this example, partial curtains 1460 are positioned at the upper region of biosensor 1400, such that each partial curtain 1460 bounds a corresponding reaction site 1414. Each partial curtain 1460 thus prevents light emitted from a corresponding reaction site 1414 from reaching image sensors 1450 that neighbor the image sensor 1450 that forms a sensing pair with the reaction site 1414

Once the emitted light exits the partial curtain 1460 (i.e., after traversing the first portion (H₂) of the height distance (H)), the emitted light continues through the layer 1432 of filter material along the second portion (H₃) of the height distance (H) and eventually reaches the image sensor 1450. The partial curtain 1460 and the layer 1432 of filter material thus cooperate to narrow the PSF of the emitted light, thereby further preventing crosstalk within the biosensor 1400.

It should be understood that the formation of partial curtains 1460 that extend along only a portion (H₂) of the height distance (H) may be simpler and less costly than the formation of curtains 540 that extend along the entire height distance (H). It should also be understood that some variations may omit the filter material of layer 1432 from the portion (H₂) of the height distance (H) through which partial curtains 1460 extend. In other words, the filter material of layer 1432 may be absent from the space defined by partial curtains 1460 under reaction sites 1414. In some such variations, this space may be filled with a different material, such as the filter material 532 described above (e.g., a filter material that is configured to absorb excitation light but not light emitted from reaction sites 1414). Alternatively, any other suitable material may be used to fill space defined by partial curtains 1460. By way of further example only, partial curtains 1460 may extend along a height of approximately 1 micrometer (while curtains 540 extend along a height of approximately 3.5 micrometers). Alternatively, partial curtains 1460 may extend along any other suitable height, provided that partial curtains 1460 do not extend along the entire height distance (H).

FIG. 20 shows a biosensor 1500 that includes a flow channel floor 1510 defining a plurality of wells 1512, with each well 1512 providing a reaction site 1514. Biosensor 1500 may be used in bioassay system 100 as a version of biosensor 102. A layer 1532 of filter material is positioned under flow channel floor 1510. A plurality of image sensors 1550 are positioned under the layer 1532 of filter material. In some versions, image sensors 1550 and layer 1532 are formed together as a single monolithic component. Each image sensor 1550 is vertically centered under a corresponding well 1512 and reaction site 1514, such that each sensor 1550 forms a sensing pair with a corresponding reaction site 1514. In this example, the layer 1532 of filter material in biosensor 1500 effectively forms a structural equivalent of base 520 in biosensor 500. The layer 1532 of filter material spans the full height distance (H) and width distance (W) of biosensor 1500. The layer 1532 of filter material in biosensor 1500 may be configured and operable like the layer 932 of filter material in biosensor 900 as described above, such that the layer 1532 of filter material may provide LICR effects as described above.

Unlike biosensor 900, and like biosensor 1400, biosensor 1500 of this example includes a plurality of partial shields or curtains 1560. Except as described below, partial curtains 1560 may be configured and operable like curtains 540 described above. Partial curtains 1560 of this are positioned between adjacent image sensors 1550 and extend through a first portion (H₂) of the height distance (H). Thus, a second portion (H₃) of the height distance (H) remains without any partial curtains 1560 extending therethrough. In other words, the layer 1532 of filter material still spans the full width distance (W) of biosensor 1500 within the second portion (H₃) of the height distance (H). In this example, partial curtains 1560 are positioned at the lower region of biosensor 1500, such that each partial curtain 1560 bounds a corresponding image sensor 1550. Each partial curtain 1560 thus prevents light emitted from a corresponding reaction site 1514 from reaching image sensors 1550 that neighbor the image sensor 1550 that forms a sensing pair with the reaction site 1514.

The light emitted from a reaction site 1514 first passes through the layer 1432 of filter material along the second portion (H₃) of the height distance (H). The emitted light then enters the space defined by the partial curtain 1460 that is under the reaction site 1514 and continues through the first portion (H₂) of the height distance (H)), eventually reaching the image sensor 1550. The partial curtain 1560 and the layer 1532 of filter material thus cooperate to narrow the PSF of the emitted light, thereby further preventing crosstalk within the biosensor 1500.

It should be understood that the formation of partial curtains 1560 that extend along only a portion (H₂) of the height distance (H) may be simpler and less costly than the formation of curtains 540 that extend along the entire height distance (H). It should also be understood that some variations may omit the filter material of layer 1532 from the portion (H₂) of the height distance (H) through which partial curtains 1560 extend. In other words, the filter material of layer 1532 may be absent from the space defined by partial curtains 1560 over image sensors 1550. In some such variations, this space may be filled with a different material, such as the filter material 532 described above (e.g., a filter material that is configured to absorb excitation light but not light emitted from reaction sites 1514). Alternatively, any other suitable material may be used to fill space defined by partial curtains 1560. By way of further example only, partial curtains 1560 may extend along a height of approximately 1 micrometer (while curtains 540 extend along a height of approximately 3.5 micrometers). Alternatively, partial curtains 1560 may extend along any other suitable height, provided that partial curtains 1560 do not extend along the entire height distance (H).

D. Examples of Biosensor with Partial Curtains and with LICR

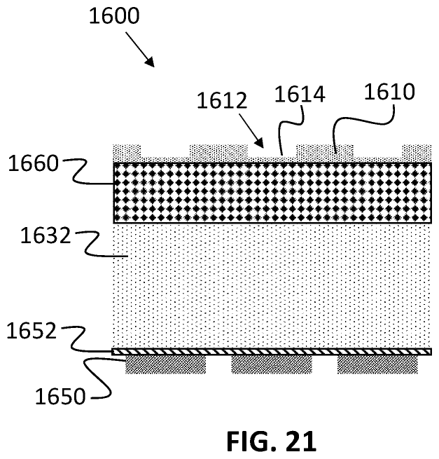

FIG. 21 shows an example of another biosensor 1600 that may be used in bioassay system 100 as a version of biosensor 102. Biosensor 1600 of this example includes a flow channel floor 1610 defining a plurality of wells 1612, with each well 1612 providing a reaction site 1614. A first optical layer 1660 is positioned under flow channel floor 1610. By way of example only, first optical layer 1660 may include tantalum pentoxide (Ta₂O₅), silicon dioxide (SiO₂), silicon nitride (Si₃N₄), and/or any other suitable material(s). First optical layer 1660 may provide additional chemical passivation, thereby effectively further sealing fluid in the flow channel of biosensor 1600 from the layer 1632 of filter material below. By way of further example only, first optical layer 1660 may have a thickness ranging from approximately 25 nm to approximately 500 nm. Alternatively, first optical layer 1660 may have any other suitable thickness. In some variations, first optical layer 1660 is omitted.

A layer 1632 of filter material is positioned under first optical layer 1660. The layer 1632 of filter material spans the full height distance width distance of biosensor 1600. The layer 1632 of filter material in biosensor 1600 may be configured and operable like the layer 932 of filter material in biosensor 900 as described above, such that the layer 1632 of filter material may provide LICR effects as described above. Examples of materials that may be used to form layer 1632 will be described in greater detail below. By way of example only, layer 1632 of filter material may have a thickness ranging from approximately 200 nm to approximately 5 μm. By way of further example only, layer 1632 of filter material may have a thickness of approximately 1 μm. Alternatively, layer 1632 of filter material may have any other suitable thickness.

In some versions, the first optical layer 1660 defines reaction sites 1614, such that layer 1632 of filter material is separated from reaction sites 1614 by the thickness of first optical layer 1660. Thus, layer 1632 of filter material may be separated from reaction sites 1614 by a distance ranging from approximately 25 nm to approximately 500 nm (or any other suitable distance). While reaction sites 1614 are provided in wells 1612 in the present example, other variations may provide reaction sites 1614 on other suitable structures, including but not limited to column structures and flat flow channel floors 1610.

A passivation layer 1652 is positioned under filter layer 1632 of filter material. By way of example only, passivation layer 1652 may include silicon dioxide (SiO₂) and/or any other suitable material(s). By way of further example only, passivation layer 1652 may have a thickness ranging from approximately 10 nm to approximately 200 nm. Alternatively, passivation layer 1652 may have any other suitable thickness. A plurality of image sensors 1650 are positioned under passivation layer 1652. While FIG. 21 shows a single passivation layer 1652 spanning continuously across all image sensors 1650, some variations may provide discrete passivation layers 1652 positioned over respective image sensors 1650, such that passivation layer 1652 need not necessarily span continuously across all image sensors 1650.

Each image sensor 1650 is vertically centered under a corresponding well 1612 and reaction site 1614, such that each sensor 1650 forms a sensing pair with a corresponding reaction site 1614. By way of example only the pitch distance between image sensors 1650 may range from approximately 0.5 μm to approximately 25 μm. By way of further example only, the pitch distance between image sensors 1650 may be approximately 1 μm. Alternatively, image sensors 1650 may have any other suitable pitch distance.

E. Examples of Biosensor with Partial Curtains and with LICR

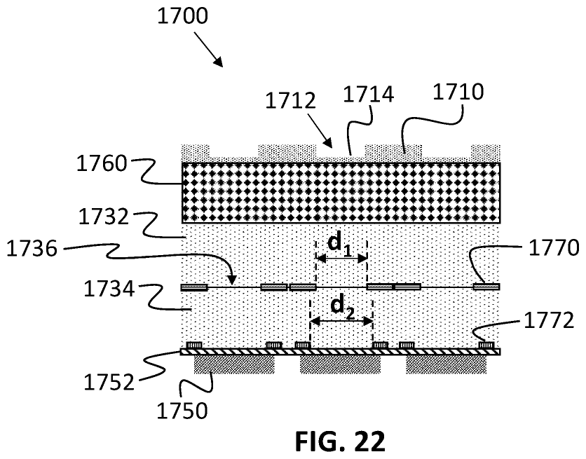

FIG. 22 shows an example of another biosensor 1700 that may be used in bioassay system 100 as a version of biosensor 102. Biosensor 1700 of this example includes a flow channel floor 1710 defining a plurality of wells 1712, with each well 1712 providing a reaction site 1714. A first optical layer 1760 is positioned under flow channel floor 1710. By way of example only, first optical layer 1760 may include tantalum pentoxide (Ta₂O₅), silicon dioxide (SiO₂), silicon nitride (Si₃N₄), and/or any other suitable material(s). First optical layer 1760 may provide additional chemical passivation, thereby effectively further sealing fluid in the flow channel of biosensor 1700 from the layer 1732 of filter material below. By way of further example only, first optical layer 1760 may have a thickness ranging from approximately 25 nm to approximately 500 nm. Alternatively, first optical layer 1760 may have any other suitable thickness. In some variations, first optical layer 1760 is omitted.

Two layers 1732, 1734 of filter material are positioned under first optical layer 1760. While two layers 1732, 1734 of filter material are provided in the present example, the two layers 1732, 1734 of filter material may be regarded as sub-layers that collectively form a layer of filter material. Thus, the terms “layer of filter material,” “optical filter layer,” and the like may be read to include arrangements that include two sub-layers like layers 1732, 1734 of filter material. In other words, layers 1732, 1734 of filter material may collectively constitute a single “layer of filter material” or “optical filter layer,” etc., as such terms are used herein. Some other variations may include more than two sub-layers of filter material collectively forming a single “layer of filter material” or “optical filter layer,” etc.

In the present example, the layers 1732, 1734 of filter material span the full height distance width distance of biosensor 1700. The layers 1732, 1734 of filter material in biosensor 1700 may together be configured and operable like the layer 932 of filter material in biosensor 900 as described above, such that the layers 1732, 1734 of filter material may together provide LICR effects as described above. Examples of materials that may be used to form layers 1732, 1734 will be described in greater detail below. By way of example only, each layer 1732, 1734 of filter material may have a thickness ranging from approximately 250 nm to approximately 250 μm. By way of further example only, each layer 1732, 1734 of filter material may have a thickness of approximately 500 nm. Alternatively, each layer 1732, 1734 of filter material may have any other suitable thickness. In the present example, the thickness of layer 1732 is approximately equal to the thickness of layer 1734. In some variations, the thickness of layer 1732 is different from the thickness of layer 1734.

In some versions, the first optical layer 1760 defines reaction sites 1714, such that layers 1732, 1734 of filter material are separated from reaction sites 1714 by the thickness of first optical layer 1760. Thus, layers 1732, 1734 of filter material may be separated from reaction sites 1714 by a distance ranging from approximately 25 nm to approximately 500 nm (or any other suitable distance). While reaction sites 1714 are provided in wells 1712 in the present example, other variations may provide reaction sites 1714 on other suitable structures, including but not limited to column structures and flat flow channel floors 1710.

A passivation layer 1752 is positioned under filter layer 1734 of filter material. By way of example only, passivation layer 1752 may include silicon dioxide (SiO₂) and/or any other suitable material(s). By way of further example only, passivation layer 1752 may have a thickness ranging from approximately 10 nm to approximately 200 nm. Alternatively, passivation layer 1752 may have any other suitable thickness. A plurality of image sensors 1750 are positioned under passivation layer 1752. While FIG. 22 shows a single passivation layer 1752 spanning continuously across all image sensors 1750, some variations may provide discrete passivation layers 1752 positioned over respective image sensors 1750, such that passivation layer 1752 need not necessarily span continuously across all image sensors 1750.

Each image sensor 1750 is vertically centered under a corresponding well 1712 and reaction site 1714, such that each sensor 1750 forms a sensing pair with a corresponding reaction site 1714. By way of example only the pitch distance between image sensors 1750 may range from approximately 0.5 μm to approximately 25 μm. By way of further example only, the pitch distance between image sensors 1750 may be approximately 1 μm. Alternatively, image sensors 1750 may have any other suitable pitch distance.

One difference between biosensor 1700 and biosensor 1600 is that biosensor 1700 includes two layers 1732, 1734 of filter material while biosensor 1600 only includes one layer 1632 of filter material. In some versions, the thickness of layer 1732 is the same as the thickness of layer 1734. By way of example only, each layer 1732, 1734 may have a thickness ranging from approximately 250 nm to approximately 2.5 μm. By way of further example only, each layer 1732, 1734 may have a thickness of approximately 500 nm. Alternatively, each layer 1732, 1734 may have any other suitable thickness. In some variations, the thickness of layer 1732 is different from the thickness of layer 1734.

Another difference between biosensor 1700 and biosensor 1600 is that biosensor 1700 includes sets of rings 1770, 1772. Each set of rings 1700, 1772 is vertically interposed between the reaction site 1714 and sensor 1750 of each sensing pair. In some versions, a vertical axis passes through the center of each reaction site 1714 and sensor 1750 of each sensing pair; and through the center of the set of rings 1770, 1772 associated with the sensing pair. Each set of rings 1770, 1772 in this example includes a first ring 1770 and a second ring 1772. First ring 1770 is positioned at an interface 1736 between the layers 1732, 1734 of filter material. Second ring 1772 is positioned between layer 1734 of filter material and passivation layer 1752. In some cases, each set of rings 1770, 1772 may function similar to partial shields or curtains 1460, 1560, such that each set of rings 1770, 1772 may effectively block optical rays between the reaction site 1714 and sensor 1750 that neighbors the sensor 1750 of the sensing pair corresponding to the set of rings 1770, 1772. Some variations may include first ring 1770 but not second ring 1772. Some other variations may include second ring 1772 but not first ring 1770. Biosensor 1600 may also be modified to include either or both of rings 1770, 1772,

Each ring 1770, 1772 comprises a metal in the present example. By way of example only, the metal may include tungsten, aluminum, or any other suitable metal (or combination of metals). By way of further example only, each ring 1770, 1772 may have a thickness of approximately 100 nm or any other suitable thickness. While each first ring 1770 has the same thickness as each second ring 1772 in the present example, each first ring 1770 may have a different thickness than each second ring 1772 in some other variations. In the present example, each first ring 1770 defines an opening with a diameter (d₁) of approximately 700 nm; while each second ring 1772 defines an opening with a diameter (d₂) of approximately 900 nm. Alternatively, each ring 1770, 1772 may define a respective opening with any other suitable diameter. In some variations, the openings defined by rings 1770, 1772 are the same, such that diameter (d₁) is equal to diameter (d₂).

It should be understood that that the diameters (d₁, d₂) associated with each set of rings 1770, 1772 may be associated with the perimeter of the pixel of the image sensor 1750 under the set of rings 1770, 1772. Moreover, the combination of rings 1770, 1772 and layers 1732, 1734 of filter material may cooperate to substantially reduce crosstalk as described herein. By way of example only, the configuration of biosensor 1600 may provide a crosstalk center fraction (i.e., the fraction of all pixel signals originating from reaction site 1614 that are recorded at the unique sensing pair pixel) of approximately 60%; while the configuration of biosensor 1700 may provide a crosstalk center fraction (i.e., the fraction of signal at the center of the pixel associated with each image sensor 1750) of approximately 70%. Alternatively, different crosstalk center fractions may be achieved, though it may be desirable for the crosstalk center fractions to enable accurate basecalling in the context of sequencing.

F. Examples of LICR Filter Materials

Any suitable material or combination of materials bay be used to form filter material of layer 932, 1432, 1532, 1632, 1732, 1734. By way of example only the filter material forming layer 932, 1432, 1532, 1632, 1732, 1734 may include a combination of a first material that is configured to provide relatively high absorption of wavelengths of excitation light 901 and a second material that is configured to provide relatively moderate absorption of wavelengths of emitted light 911. In some versions of this example, the first material is configured to substantially absorb light at wavelengths below about 500 nm; and to not substantially absorb light at wavelengths above about 600 nm. In addition, in some versions of this example, the second material is configured to substantially absorb light at wavelengths below about 600 nm; and to not substantially absorb light at wavelengths above about 600 nm. By way of further example only, the combination may include approximately 0.1 ppm to approximately 1% of the second material blended with the first material. Such a combination may provide absorption at about 10⁷ for wavelengths around 600 nm.

In some versions where a combination of materials is used to form filter material of layer 932, 1432, 1532, 1632, 1732, 1734, providing absorption at about 10⁷ for wavelengths around 600 nm, the first material of the combination includes an orange organic dye while the second material of the combination includes a black organic dye. By way of example only, a combination of materials as described above to form filter material of layer 932, 1432, 1532, 1632, 1732, 1734 may be particularly suitable for contexts where image sensors 950, 1450, 1550 a relatively large pixel pitch (e.g., greater than approximately 3 μm).

As another example, the filter material of layer 932, 1432, 1532, 1632, 1732, 1734 may include ferric oxide (Fe₂O₃). In some scenarios, ferric oxide may be particularly suitable for contexts where image sensors 950, 1450, 1550 a relatively small pixel pitch (e.g., approximately 2 μm, between approximately 2 μm and approximately 1 μm, or less than approximately 1 μm). By way of further example only, including ferric oxide in the filter material of layer 932, 1432, 1532, 1632, 1732, 1734 may be particularly suitable for contexts where red fluorophores are used in biosensor 900, 1400, 1500. In some versions where the filter material of layer 932, 1432, 1532 includes ferric oxide, layer 932, 1432, 1532, 1632, 1732, 1734 may substantially absorb light at wavelengths below about 550 nm; moderately absorb light at wavelengths between about 550 nm and about 700 nm; and weakly absorb light at wavelengths between about 760 nm and about 1,500 nm. By substantially blocking (e.g., providing transmission of less than 0.1%) excitation light at wavelengths below about 550 nm, providing moderate transmission of light in a wavelength range between about 600 nm and about 700 nm, and providing substantial transmission of light in a wavelength range above about 700 nm, a filter layer 932, 1432, 1532 including ferric oxide may effectively provide LICR, particularly with respect to red fluorophores.

Regardless of whether the filter material of layer 932, 1432, 1532, 1632, 1732, 1734 includes a combination of orange organic dye and black organic dye, ferric oxide, and/or other materials, the material(s) may be applied as a layer over image sensors 950, 1450, 1550 having any suitable thickness. By way of example only, the thickness may range from approximately 100 nm to approximately 15 μm; or may be approximately 1 μm.

Alternatively, any other suitable materials and combinations may be used to form filter material of layer 932, 1432, 1532, 1632, 1732, 1734, with materials being selected based on criteria including (but not necessarily limited to) the wavelength of the excitation light 901 and the wavelength of the emitted light 911.

E. Examples of Other Features and Variations

In some of the various examples provided above, image sensors 440, 550, 650, 950, 1450, 1550 are configured and arranged such that image sensors 440, 550, 650, 950, 1450, 1550 provide a single pixel per reaction site 414, 514, 614, 914, 1414, 1514. In other words, the pixel-to-reaction site ratio is 1:1. Since each reaction site 414, 514, 614, 914, 1414, 1514 is defined in a single corresponding well 408, 512, 612, 912, 1412, 1512 in the various examples provided above, the pixel-to-well ratio may also be 1:1. However, in some other variations, image sensors 440, 550, 650, 950, 1450, 1550 are configured and arranged such that the pixel-to-well ratio or pixel-to-reaction site ratio is greater than 1:1. In other words, some alternative configurations may provide two or more wells or reactions sites per pixel. Any of the teachings herein may be applied to such alternative configurations providing two or more wells or reactions site per pixel.

In some versions providing two or more wells or reaction site per pixel, selective illumination may be applied to selectively illuminate the two or more wells or reaction sites sharing a single pixel. Selective illumination may include illuminating one well or reaction site of the shared single pixel at one moment in time, then subsequently illuminating another well or reaction site of the same shared single pixel at a subsequent moment in time. Such selective illumination may be provided by selectively applying shutters, moving the light source relative to the wells or reaction sites, moving the reaction sites relative to the wells, or in any other suitable fashion. By way of further example only, such selective illumination may be provided in accordance with at least some of the teachings of U.S. Pub. No. 2019/0212295, entitled “Systems and Devices for High-Throughput Sequencing with Semiconductor-Based Detection,” published Jul. 11, 2019, the disclosure of which is incorporated by reference herein, in its entirety. The teachings herein may also be combined with various teachings of U.S. Pub. No. 2019/0170904, entitled “Photonic Structure-Based Devices and Compositions for Use in Luminescent Imaging of Multiple Sites within a Pixel, and Methods of Using the Same,” published Jun. 6, 2019, the disclosure of which is incorporated by reference herein, in its entirety.

Alternatively, intensity multiplexing may be used provide illumination and optical sensing in arrangements providing two or more wells or reactions site per pixel. By way of example only, such multiplexing may be provided in accordance with at least some of the teachings of U.S. Provisional Pat. App. No. 63/200,383, entitled “Sensor with Multiple Reaction Sites per Pixel,” filed Mar. 3, 2021, the disclosure of which is incorporated by reference herein, in its entirety.

IV. Miscellaneous

It is to be understood that the subject matter described herein is not limited in its application to the details of construction and the arrangement of components set forth in the description herein or illustrated in the drawings hereof. The subject matter described herein is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

When used in the claims, the term “set” should be understood as one or more things which are grouped together. Similarly, when used in the claims “based on” should be understood as indicating that one thing is determined at least in part by what it is specified as being “based on.” Where one thing is required to be exclusively determined by another thing, then that thing will be referred to as being “exclusively based on” that which it is determined by.

Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings. Also, it is to be understood that phraseology and terminology used herein with reference to device or element orientation (such as, for example, terms like “above,” “below,” “front,” “rear,” “distal,” “proximal,” and the like) are only used to simplify description of one or more examples described herein, and do not alone indicate or imply that the device or element referred to must have a particular orientation. In addition, terms such as “outer” and “inner” are used herein for purposes of description and are not intended to indicate or imply relative importance or significance.

It is to be understood that the above description is intended to be illustrative, and not restrictive. For example, the above-described examples (and/or aspects thereof) may be used in combination with each other. In addition, many modifications may be made to adapt a particular situation or material to the teachings of the presently described subject matter without departing from its scope. While the dimensions, types of materials and coatings described herein are intended to define the parameters of the disclosed subject matter, they are by no means limiting and instead illustrations. Many further examples will be apparent to those of skill in the art upon reviewing the above description. The scope of the disclosed subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

The following claims recite aspects of certain examples of the disclosed subject matter and are considered to be part of the above disclosure. These aspects may be combined with one another.

What is claimed is:

1. An apparatus, comprising:
a flow cell body defining a channel to receive fluid, the channel having a floor extending along a length of the flow cell body;
a plurality of reaction sites positioned along the floor of the channel, the plurality of reaction sites forming an array along a length of the floor of the channel;
an optical filter layer positioned under the floor of the channel, the optical filter including at least a portion spanning uninterruptedly along a length corresponding to the length of the array of reaction sites; and
a plurality of imaging regions positioned under the optical filter layer, each imaging region of the plurality of imaging regions being positioned directly under a corresponding reaction site, such that each reaction site and corresponding imaging region cooperate to form a sensing pair;
the optical filter layer permits one or more selected wavelengths of light to pass from each reaction site to the imaging region forming a sensing pair with the reaction site;
the optical filter layer reduces transmission of excitation light directed toward the plurality of reaction sites, the optical filter layer reduces transmission of light emitted from each reaction site to imaging regions not forming a sensing pair with the reaction site.

2. The apparatus of claim 1, the floor of the channel defining a plurality of wells, the plurality of wells providing the plurality of reaction sites.

3. The apparatus of claim 1, the flow cell body defining a plurality of channels, the channels being oriented parallel with each other, each channel of the plurality of channels having a floor with a plurality of reaction sites.

4. The apparatus of claim 3, the plurality of channels forming an array along a width of the flow cell body, the optical layer including at least a portion spanning uninterruptedly along a width corresponding to the width of the array of channels.

5. The apparatus of claim 1, further comprising a plurality of imaging sensors, each imaging sensor forming a corresponding imaging region of the plurality of imaging regions.

6. The apparatus of claim 1, the optical filter layer reduces transmission of light from each reaction site to imaging regions not forming a sensing pair with the reaction site by inducing loss in light transmitted from the reaction sites.

7. The apparatus of claim 1, further comprising a plurality of shields, each shield of the plurality of shields to block optical rays between a corresponding reaction site and an imaging region of the plurality of imaging regions that does not form a sensing pair with the corresponding reaction site.

8. The apparatus of claim 1, the optical filter layer substantially prevents transmission of light at wavelengths less than approximately 500 nm, the optical filter layer absorbs some light at wavelengths between approximately 500 nm and approximately 600 nm while permitting transmission of some light at wavelengths between approximately 500 nm and approximately 600 nm.

9. The apparatus of claim 1, the optical filter layer including a combination of an orange dye and a black dye.

10. The apparatus of claim 1, the optical filter layer having a transmittance coefficient ranging from approximately 0.1 to approximately 0.5.

11. The apparatus of claim 1, the optical filter layer and floor cooperating to define a height dimension, the height dimension corresponding to a distance between a top of the floor and a bottom of the optical filter layer,
the plurality of reaction sites defining a pitch dimension, the pitch dimension corresponding to a distance between a center of one reaction site of the plurality of reaction sites to a center of an adjacent reaction site of the plurality of reaction sites,
the height dimension and pitch dimension providing a height-to-pitch ratio ranging from approximately 3 to approximately 5.

12. The apparatus of claim 1, the apparatus lacking any shields between the plurality of reaction sites and the plurality of imaging regions.

13. The apparatus of claim 1, the optical filter layer having a thickness ranging from approximately 500 nm to approximately 5 μm.

14. The apparatus of any of claim 1, the imaging regions being separated from each other by a pitch distance ranging from approximately 0.5 μm to approximately 25 μm.

15. The apparatus of claim 1, the optical filter layer comprising a first sub-layer of filter material and a second sub-layer of filter material.

16. The apparatus of any of claim 15, further comprising a plurality of rings, the plurality of rings being positioned adjacent to one or both of the first sub-layer of filter material or the second sub-layer of filter material.

17. The apparatus of claim 16, the plurality of rings including a first array of rings and a second array of rings, the first array of rings being located at a first vertical position between the reaction sites and the plurality of imaging regions, the second array of rings being located at a second vertical position between the reaction sites and the plurality of imaging regions.

18. The apparatus of claim 1, the optical filter layer including ferric oxide.

19. A method of manufacturing a flow cell, the method comprising:
forming an optical filter layer over an imaging layer, the imaging layer defining a plurality of imaging regions, the imaging layer extending along a first length, the imaging layer being operable to capture images at the plurality of imaging regions; the optical filter layer extending continuously along the first length;
positioning a floor over the optical filter layer, the floor extending along the first length of the flow cell, the floor defining a plurality of reaction sites over the optical filter layer, the plurality of reaction sites forming an array along the first length such that the optical filter layer extends continuously along a region under all the reaction sites of the plurality of reaction sites, each reaction site of the plurality of reaction sites being positioned directly over a corresponding imaging region of the plurality of imaging regions such that each reaction site cooperates with a corresponding imaging region to form a sensing pair; and
positioning a cover over the floor, the floor and the cover cooperating to define a fluid channel, the fluid channel extending along the first length;
the cover, the floor, the optical filter layer, and the imaging layer cooperating to form at least a portion of a flow cell body;
the optical filter layer permits one or more selected wavelengths of light to pass from each reaction site to the imaging region forming a sensing pair with the reaction site;
the optical filter layer reduces transmission of excitation light directed toward the plurality of reaction sites, the optical filter layer reduces transmission of light emitted from each reaction site to imaging regions not forming a sensing pair with the reaction site.

20. An apparatus, comprising:
a flow cell body defining a channel to receive fluid, the channel having a floor extending along a length of the flow cell body;
a plurality of reaction sites positioned along the floor of the channel, the plurality of reaction sites forming an array along a length of the floor of the channel;
an optical filter layer positioned under the floor of the channel, the optical filter including at least a portion spanning uninterruptedly along a length corresponding to the length of the array of reaction sites; and
a plurality of imaging regions positioned under the optical filter layer, each imaging region of the plurality of imaging regions being positioned directly under at least one corresponding reaction site of the plurality of reaction sites, such that each reaction site and corresponding imaging region cooperate to form a sensing relationship;
the optical filter layer being configured to permit one or more selected wavelengths of light to pass from each reaction site to the imaging region forming a sensing relationship with the reaction site;
the optical filter layer being configured to reduce transmission of excitation light directed toward the plurality of reaction sites, the optical filter layer being further configured to reduce transmission of light emitted from each reaction site to imaging regions not forming a sensing relationship with the reaction site.
